SEQUENCE LISTING (24 sequences)

SEQ ID NO: 1 - DNA, 2440 bp, synthetic construct
  misc_feature 1..2440: note = miR451 scaffold expression construct
  source 1..2440: mol_type = other DNA
cagtcacgacgttgtaaaacgacggccagtgaattcgcccttcaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgcttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaagggcgaattcgagctcggtacctcgcgaatgcatctagatatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtctggaactaccttgcatacttatgcaaggtagttccagtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatccagatctcgagcggccgcccgtggcatccctgtgacccctccccagtgcctctcctggccctggaagttgccactccagtgcccaccagccttgtcctaataaaattaagttgcatcaagatcgacgggcccgtcgactgcagag

SEQ ID NO: 2 - DNA, 22 bp, Homo sapiens
  source 1..22: mol_type = other DNA
tctggaactaccttgcatactt

SEQ ID NO: 3 - DNA, 22 bp, Homo sapiens
  source 1..22: mol_type = other DNA
aaggacttgagggactcgaaga

SEQ ID NO: 4 - DNA, 72 bp, synthetic construct
  misc_feature 1..72: note = HTT miRNA encoding sequence
  source 1..72: mol_type = other DNA
cttgggaatggcaaggaaggacttgagggactcgaagacgagtccctcaagtcctctcttgctatacccaga

SEQ ID NO: 5 - RNA, 22 nt, synthetic construct
  misc_feature 1..22: note = RNA sequences complementary to HTT
  source 1..22: mol_type = other RNA
aaggacttgagggactcgaaga

SEQ ID NO: 6 - RNA, 22 nt, synthetic construct
  misc_feature 1..22: note = RNA sequences complementary to ATNX3
  source 1..22: mol_type = other RNA
tctggaactaccttgcatactt

SEQ ID NO: 7 - DNA, 24 bp, synthetic construct
  misc_feature 1..24: note = Small RNAseq analysis of sequences produced from the scaffold targeting Huntington
  source 1..24: mol_type = other DNA
aaggacttgagggactcgaagacg

SEQ ID NO: 8 - DNA, 30 bp, synthetic construct
  misc_feature 1..30: note = Small RNAseq analysis of sequences produced from the scaffold targeting Huntington
  source 1..30: mol_type = other DNA
aaggacttgagggactcgaagacgagtccc

SEQ ID NO: 9 - DNA, 23 bp, synthetic construct
  misc_feature 1..23: note = Small RNAseq analysis of sequences produced from the scaffold targeting Huntington
  source 1..23: mol_type = other DNA
aaggacttgagggactcgaagac

SEQ ID NO: 10 - DNA, 25 bp, synthetic construct
  misc_feature 1..25: note = Small RNAseq analysis of sequences produced from the scaffold targeting Huntington
  source 1..25: mol_type = other DNA
aaggacttgagggactcgaagacga

SEQ ID NO: 11 - DNA, 21 bp, synthetic construct
  misc_feature 1..21: note = Small RNAseq analysis of sequences produced from the scaffold targeting Huntington
  source 1..21: mol_type = other DNA
aaggacttgagggactcgaag

SEQ ID NO: 12 - DNA, 27 bp, synthetic construct
  misc_feature 1..27: note = Small RNAseq analysis of sequences produced from the scaffold targeting Huntington
  source 1..27: mol_type = other DNA
aaggacttgagggactcgaagacgagt

SEQ ID NO: 13 - DNA, 19 bp, synthetic construct
  misc_feature 1..19: note = Small RNAseq analysis of sequences produced from the scaffold targeting Huntington
  source 1..19: mol_type = other DNA
aaggacttgagggactcga

SEQ ID NO: 14 - DNA, 31 bp, synthetic construct
  misc_feature 1..31: note = Small RNAseq analysis of sequences produced from the scaffold targeting Huntington
  source 1..31: mol_type = other DNA
aaggacttgagggactcgaagacgagtccct

SEQ ID NO: 15 - DNA, 20 bp, synthetic construct
  misc_feature 1..20: note = Small RNAseq analysis of sequences produced from the scaffold targeting Huntington
  source 1..20: mol_type = other DNA
aaggacttgagggactcgaa

SEQ ID NO: 16 - DNA, 24 bp, synthetic construct
  misc_feature 1..24: note = Small RNAseq analysis of tsequences produced from the scaffold targeting ATXN3
  source 1..24: mol_type = other DNA
tctggaactaccttgcatacttat

SEQ ID NO: 17 - DNA, 23 bp, synthetic construct
  misc_feature 1..23: note = Small RNAseq analysis of tsequences produced from the scaffold targeting ATXN3
  source 1..23: mol_type = other DNA
tctggaactaccttgcatactta

SEQ ID NO: 18 - DNA, 21 bp, synthetic construct
  misc_feature 1..21: note = Small RNAseq analysis of tsequences produced from the scaffold targeting ATXN3
  source 1..21: mol_type = other DNA
tctggaactaccttgcatact

SEQ ID NO: 19 - DNA, 30 bp, synthetic construct
  misc_feature 1..30: note = Small RNAseq analysis of tsequences produced from the scaffold targeting ATXN3
  source 1..30: mol_type = other DNA
tctggaactaccttgcatacttatgcaagg

SEQ ID NO: 20 - RNA, 16 nt, synthetic construct
  misc_feature 1..16: note = miRNA451 scaffold 5'sequence
  source 1..16: mol_type = other RNA
cttgggaatggcaagg

SEQ ID NO: 21 - RNA, 17 nt, synthetic construct
  misc_feature 1..17: note = miRNA451 scaffold 3'sequience
  source 1..17: mol_type = other RNA
ctcttgctatacccaga

SEQ ID NO: 22 - RNA, 71 nt, synthetic construct
  misc_feature 1..71: note = Pre-miC_451
  variation 17..55: note = n is a, c, g, or u
  source 1..71: mol_type = other RNA
cttgggaatggcaaggnnnnnnnnnnnnnnnnnnnnnnnnnnnnnnnnnnnnnnntcttgctatacccaga

SEQ ID NO: 23 - DNA, 16 bp, synthetic construct
  misc_feature 1..16: note = forward primer sequence
  source 1..16: mol_type = other DNA
gagccgcagccattgc

SEQ ID NO: 24 - DNA, 23 bp, synthetic construct
  misc_feature 1..23: note = reverse primer sequence
  source 1..23: mol_type = other DNA
cacagatttgggacaaaggaagt

(19) United States
(12) Patent Application Publication
(10) Pub. No.: US 2026/0139305 A1
(43) Pub. Date: May 21, 2026

(54) COMPANION DIAGNOSTIC TO MONITOR THE EFFECTS OF GENE THERAPY

(71) Applicant: uniQure IP B.V., Amsterdam (NL)

(72) Inventors: Astrid Valles-Sanchez, Amsterdam (NL); Pavlina Stefanova Konstantinova, Amsterdam (NL)

(73) Assignee: uniQure IP B.V., Amsterdam (NL)

(21) Appl. No.: 19/327,350

(22) Filed: Sep. 12, 2025

Related U.S. Application Data
(63) Continuation of application No. 17/323,641, filed on May 18, 2021.
Application No. 17/323,641 is a continuation of application No. PCT/EP2019/081759, filed on Nov. 19, 2019.
(60) Provisional application No. 62/769,108, filed on Nov. 19, 2018.

(30) Foreign Application Priority Data
Nov. 19, 2018 (EP) 18206967.4

(51) Int. Cl. C12Q 1/686; C12Q 1/6883

(52) U.S. Cl. CPC C12Q 1/686; C12Q 1/6883; C12Q 2600/158; C12Q 2600/178

(57) ABSTRACT

The invention relates to the field of gene therapy. In addition the invention relates to the field of interfering RNA and/or microRNA (miRNA). In particular the invention relates to gene therapy involving such miRNA's and more in particular to diagnostic tools to be implemented in the treatment of disease, when such treatment is carried out by delivery of miRNA's to a patient, i.e. to cells of a patient. The present invention uses the content of extracellular vesicles that are found in body fluids to determine the presence of miRNA's or the like expressed through the introduction of a gene delivery vehicle is in a host.

The invention relates to the field of gene therapy. In addition the invention relates to the field of interfering RNA and/or microRNA (miRNA). In particular the invention relates to gene therapy involving such miRNA's and more in particular to diagnostic tools to be implemented in the treatment of disease, when such treatment is carried out by delivery of miRNA's to a patient, i.e. to cells of a patient. More in particular the invention relates to the treatment of neurodegenerative diseases such as Huntington's disease and monitoring the effects of treatment of such diseases.

### REFERENCE TO SEQUENCE LISTING SUBMITTED ELECTRONICALLY

The content of the electronically submitted sequence listing (Name: 5748_0060004_Sequencelisting_ST26.xml; Size: 34,550 bytes; and Date of Creation: Dec. 1, 2025), is incorporated herein by reference in its entirety.

### BACKGROUND

Gene Therapy The elucidation of DNA as the carrier of genetic information, and therefore also as the source of inherited diseases, has led to envisaged therapies in which mutant, damaged genes could be replaced or silenced. Many genes and/or other nucleic acid sequences have now been identified to play a role in (genetic) disease. If the mutant gene(s) could be replaced by a healthy one, or if the genes expressing aberrant (sometimes toxic) products could be silenced, the disease could be treated at the molecular level, and, potentially be cured. Gene therapy provides a promising concept, in particular for diseases caused by mutations in a single gene.

However, the delivery of the desired nucleic acid to the cells that need to be targeted is not an easy task. Numerous (viral) delivery systems have been investigated, all of them with their own advantages and drawbacks. One of the viral delivery vehicles that is used for gene therapy is Adeno Associated Virus (AAV).

### AAV.

AAV has a single-stranded DNA genome of approximately 4.8 kilobases (kb). AAV belongs to the parvovirus family and is dependent for replication on co-infection with other viruses, in particular adenoviruses. The genome comprises, Rep (Replication) and Cap (Capsid) genes. These coding sequences are flanked by inverted terminal repeats (ITRs) that are required for genome replication and packaging. The Rep gene encodes four proteins (Rep78, Rep68, Rep52, and Rep40), replicate the viral genome and faciliate packaging, while Cap expression gives rise to the viral capsid proteins (VP; VP1/VP2/VP3), which form the outer capsid shell.

For gene therapy the viral DNA of the AAV is almost completely removed. Recombinant AAV (rAAV) for gene therapy is formed by a protein capsid containing a desired nucleic acid, the transgene, that is to be delivered to target cells. The desired nucleic acid is flanked with the ITR's of AAV. ITR-flanked transgenes encoded by rAAV can form circular concatemers remaining in the nucleus of transduced cells as episomes. As the episome remains largely episomal, the expression of AAV delivered nucleic acid sequences may be diluted over time if and when the target cell replicates. This dilution may not generally apply to post-mitotic cells such as neurons, which are the target cells for many neurodegenerative diseases. A recent review on AAV vectors for gene therapy is provided in Naso et al, Biodrugs 2017 (p.317-334).

miRNA

RNA interference (RNAi) is a naturally occurring mechanism that involves sequence specific down regulation of messenger RNA (mRNA). The down regulation of mRNA results in a reduction of the amount of protein that is expressed. RNA interference is triggered by double stranded RNA. One of the strands of the double stranded RNA is substantially or completely complementary to its target, the mRNA. This strand is termed the guide strand. The mechanism of RNA interference involves the incorporation of the guide strand in the RNA-induced silencing complex (RISC). This complex is a multiple turnover complex that via complementary base paring of the guide strand can bind to its target mRNA. Once bound to its target mRNA it can either cleave the mRNA or reduce translation efficiency. RNA interference has since its discovery been widely used to knock down specific target genes. The triggers for inducing RNA interference that have been employed involve the use of small interfering RNAs (siRNAs) or short hairpin RNAs (shRNAs). In addition, molecules that can naturally trigger RNAi, the so called micro RNAs (miRNAs), have been used to make artificial (engineered) miRNAs that mimic their naturally occurring counterparts. These strategies have in common that they provide for substantially double stranded RNA molecules that are designed to target a mRNA of choice. RNAi based therapeutic approaches that utilise the sequence specific modality of RNAi are under development and several are currently in clinical trials (see i.a. Davidson and McCray, Nature Reviews—Genetics, 2011; Vol. 12; 329-340).

### Huntington's Disease

The huntingtin gene, also referred to as the HTT or HD (Huntington's disease) gene, encodes for the huntingtin mRNA and protein. The huntingtin gene is a large gene on chromosome 4p.13 of about 13.5kb (huntingtin protein is about 350 kDa). Huntington's disease is a genetic neurodegenerative disorder caused by a genetic mutation in the huntingtin gene. The genetic mutation involves a DNA segment in exon 1 of the huntingtin gene known as the CAG trinucleotide repeat. Normally, the CAG segment in the huntingtin gene of humans is repeated multiple times, i.e. about 10-35 times. People that develop Huntington's disease have an expansion of the number of CAG repeats in at least one allele. An affected person usually inherits the mutated allele from one affected parent. In rare cases, an individual with Huntington's disease does not have a parent with the disorder (sporadic HD). People with 36 to 39 CAG repeats may develop signs and symptoms of HD, while people with 40 or more repeats always develop the disorder, marked by a triad of motor, cognitive and psychiatric symptoms that ultimately leads to death. The increase in the size of the CAG repeat leads to the production of a an aberrant HTT mRNA resulting in an RNA toxic gain-of-function, and to a production of mutant huntingtin protein with an elongated polyglutamine (polyQ) stretch. The mutant huntingtin protein is processed in the cell into smaller fragments that are cytotoxic and that accumulate and aggregate in neurons, starting in the striatum and in the cerebral cortex in later stages of the disease. This results in the disruption of normal function and eventual death of neurons. This is the main process that occurs in the brain which underlies the signs and symptoms of Huntington's disease.

### SCA3

Spinocerebellar ataxia type 3 (SCA3), or Machado-Joseph disease (MID), is an autosomal dominant monogenic, fatal disorder. The disorder is characterized by progressive degeneration of brain areas, which is caused by a CAG expansion in the human ataxin-3 gene, also referred to as ATXN3 gene (OMIM: 607047, reference sequence Homo sapiens ataxin 3 (ATXN3) on chromosome 14, NCBI Reference Sequence: NG_008198.2. In the 3′ region of the gene, a cytosine-adenine-guanine (CAG) repeat region is present. Said CAG region is in frame and results in an ataxin-3 protein comprising a polyQ region, a repetitive sequence of glutamines. Healthy, or non-symptomatic, individuals may have up to 44 CAG-repeats in the ATXN3 gene. Diseased individuals have expansions and it has been shown that they may have between 52 and 86 or more CAG repeats. Individuals having between 45-51 CAG repeats are to have symptoms with incomplete penetrance of disease. Said expansion resulting in ataxin-3 protein that have extended polyQ regions and the length of the CAG repeats, and thus polyQ regions within ataxin-3, can be correlated with disease progression, i.e. the longer the region usually the more progressive the disease.

The ataxin-3 protein with the expanded polyQ tract acquires toxic properties (gain of toxic function) and the formation of neuronal aggregates in the brain is the neuropathological hallmark. Neuropathological studies have detected widespread neuronal loss in various areas, including cerebellum, thalamus, midbrain, pons, medulla oblongata and spinal cord of SCA3 patients (Riess et al., Cerebellum 2008). Although widespread pathology is reported, the consensus is that the main pathology is in the cerebellum and in the brainstem (Eichler et al. AJNR Am J Neuroradiol, 2011). The disease has full penetration, which means that if a person has an expansion of 52 or more CAGs, they will inevitably develop the disease and have 50% chance to pass it on to their offspring.

### SUMMARY OF THE INVENTION

As disclosed above the expression of a desired product for the purpose of gene therapy may decrease or even disappear over time. It may therefore be desirable to be able to determine whether the desired product is still expressed by cells in the host. In many cases it may not be easy or even possible to determine expression in target cells themselves. In particular when the desired product is delivered to a less accessible organ such as the brain, or if the product produced from the introduction of the gene delivery vehicle is not found in other body fluids. The present invention uses the content of extracellular vesicles that are found in body fluids to determine the presence of the product expressed through the introduction of a gene delivery vehicle is in a host. In particular the invention provides methods and means for the detection of artificial miRNAs in extracellular vehicles. More in particular the invention provides methods and means for the detection and/or quantification of artificial miRNAs expressed through the introduction of a gene delivery vehicle, in particular an AAV based vehicle (an AAV vector). The methods and means of the invention are particularly useful for the detection/determination of expression of an artificial miRNA introduced by gene therapy intended for the treatment of neurodegenerative diseases, in particular Huntington's disease or spinocerebellar ataxia type 3 (SCA3).

### Embodiments

In one embodiment the invention provides a method for determining expression of at least one artificial miRNA in the human body, comprising providing a sample of a body fluid, e.g. cerebrospinal fluid (CSF) or serum/plasma from a patient having been treated with said AAV delivered artificial miRNA and determining the abundance of said artificial miRNA in extracellular vesicles containing at least part of said artificial miRNA in said CSF or serum/plasma sample. In the context of the present invention the term artificial means made or introduced by man (direct or indirect) or altered from nature. According to the invention the expression of the expressed product introduced by a gene delivery vehicle will lead to incorporation of said expressed product (typically an artificial miRNA) in extracellular vesicles. By determining the presence of the product introduced by a gene delivery vehicle in a body fluid, the expression of the product in target cells or target tissue can be monitored.

According to the invention the expression of the product introduced by a gene delivery vehicle will lead to incorporation of said product (typically an artificial miRNA) in extracellular vesicles. By determining the presence of the expressed product introduced by a gene delivery vehicle in a body fluid, the expression of the product in target cells can be monitored. According to the invention said expression of the product will be preferably in primates, such as humans. The introduction of the gene delivery vehicle will lead to incorporation of its product (typically an artificial miRNA) in extracellular vesicles. Hence, preferably, said gene delivery vehicle is most preferably for use in primates, such as humans, and expressed miRNAs from a transgene are thus preferably monitored in body fluids of humans. By determining the presence of the expressed product introduced by a gene delivery vehicle in a body fluid, such as of a human, the expression of the product in target cells can be monitored. Suitable body fluids may be the CSF or blood, such as serum and plasma. According to the invention CSF is preferred.

In one embodiment the invention provides a method for determining expression of at least one artificial miRNA in the central nervous system, such as the brain and/or spinal cord, comprising providing a sample of CSF from a patient having been treated with said AAV delivered artificial miRNA and determining the abundance of said artificial miRNA in extracellular vesicles containing at least part of said artificial miRNA in said CSF.

In one embodiment the invention provides a method for determining expression of at least one artificial miRNA in the central nervous system, such as the brain and/or spinal cord, comprising providing a sample of serum/plasma from a patient having been treated with said AAV delivered artificial miRNA and determining the abundance of said artificial miRNA in extracellular vesicles containing at least part of said artificial miRNA in said serum/plasma sample.

According to the invention the preferred extracellular vesicles are exosomes, although microvesicles can also be considered. Extracellular vesicles according to the invention are characterized by their size (exosomes ranging between 30-100-nm in diameter, and microvesicles being generally larger, between 100-1000 nm in diameter), the presence on their surface of certain markers, such as exosomal markers CD9, CD63, CD61, TSG101 and Alix, and microvesicle markers such as integrins, selectins (CD62), CD40 ligand and CD133 and the extracellular vesicle preparation further characterized by e.g. the presence of RNA-carrying contaminants such as ribonucleoprotein complexes (RNPs), viral particles, and lipoproteins (HDL and LDL). According to the invention any method of enriching a biological sample in extracellular vesicles and determining the presence of the product, in particular the artificial miRNA may be employed. It is not necessary to determine the presence of the whole product, a characterizing part may very well be sufficient. Although quantification of the presence of the product (the artificial miRNA) is preferred, absolute quantification may not be necessary. Detectable presence of the product in extracellular vesicles may be sufficient, but also semi-quantified information may be enough to determine continuing expression of the gene delivery vehicle's product.

Furthermore, said method determining of expression of at least one may miRNA may also occur through further means. For example, as shown in the example section, miRNAs may not only associated with extracellular vesicles, but are also found in fractions associated with proteins and lipoproteins, such a high density lipoproteins (HDLP). Hence, accordingly said the methods herein may also utilize such fractions of proteins, lipoproteins or HDLP comprising said miRNA.

Apart from the method for determining (and/or detecting) the artificial miRNA in a sample, the invention also provides means for carrying out such determinations, typically in the form of a kit comprising the necessary reagents to isolate and purify extracellular vesicles, in particular exosomes and microvesicles; reagents to isolate and/or detect/quantify artificial miRNAs, including primers and probes and enzymes and the like; reagents to serve as a reference or reagents that produce an internal reference, optionally secondary detection means such as labels, buffers etc.

According to the invention an artificial miRNA is defined as a sequence that results from replacing a miRNA sequence found in nature with another sequence as it is comprised in its precursor, i.e. a pre-miRNA or a pri-miRNA sequence. The miRNA produced from said scaffold no longer being the miRNA as found in nature (not the original sequence, hence the term artificial). MicroRNAs, i.e. miRNA, are guide strands that originate from double stranded RNA molecules that are endogenously expressed e.g. in mammalian cells. A miRNA is processed from a pre-miRNA precursor molecule by the RNAi machinery and incorporated in an activated RNA-induced silencing complex (RISC) (Tijsterman M, Plasterk R H, Cell. 2004 Apr. 2; 1 17(1):1-3). A pre-miRNA is a hairpin RNA molecule that can be part of a larger RNA molecule (pri-miRNA), e.g. comprised in an intron, which is first processed by Drosha to form a pre-miRNA hairpin molecule. The pre-miRNA molecule is an shRNA-like molecule that can subsequently be processed by Dicer to result in an siRNA-like double stranded RNA duplex. One endogenous miRNA, miR451 does not require Dicer for processing, but it is instead processed by the Argonaute 2 (Ago2) enzyme and subsequently trimmed by the Poly(A)-specific ribonuclease (PARN) to the mature 22/26-nt miR451 (Herrera-Carrillo and Berkhout, Nucleic Acids Res, 2017, 45(18):10369-10379). The resulting mature miRNA, obtained either by Dicer-dependent or by Dicer-independent processing, i.e. the guide strand, that is part of the double stranded RNA duplex is subsequently incorporated in RISC. An RNA molecule such as present in nature, i.e. a pri-miRNA, a pre-miRNA or a miRNA duplex, may be used as a scaffold for producing an artificial miRNA that specifically targets a gene of choice. Based on the predicted RNA structure of the RNA molecule as present in nature, e.g. as predicted using e.g. m-fold software using standard settings (Zuker. Nucleic Acids Res. 31(13), 3406-3415, 2003), the natural miRNA sequence as it is present in the RNA structure (i.e. duplex, pre-miRNA or pri-miRNA), and the sequence present in the structure that is substantially complementary therewith are removed and replaced with a first RNA sequence and a second RNA sequence. The first RNA sequence and the second RNA sequence are preferably selected such that the predicted secondary RNA structures that are formed, i.e. of the pre-miRNA, pri-miRNA and/or miRNA duplex, resemble the corresponding predicted original secondary structure of the natural RNA sequences. pre-miRNA, pri-miRNA and miRNA duplexes (that consist of two RNA strands that are hybridized via complementary base pairing) as found in nature often are not fully base paired, i.e. not all nucleotides that correspond with the first and second strand as defined above are base paired, and the first and second strand are often not of the same length. How to use miRNA precursor molecules as scaffolds for any selected target RNA sequence and substantially complementary first RNA sequence is described e.g. in Liu Y P Nucleic Acids Res. 2008 May; 36(9):281 1-24, which is incorporated herein by reference.

Whatever design is used for the miRNA scaffold, which is preferably based on miR451, it is designed such that therefrom an antisense RNA molecule comprising the first RNA sequence, i.e. the sequence that replaced the original miRNA sequence, in whole or a substantial part thereof, can be processed by the RNAi machinery such that it is incorporated in the RISC complex to have its action, i.e. to induce RNAi e.g. against the RNA target sequence comprised in an RNA encoded by gene associated with a disease. The artificial miRNA that is produced from the miRNA scaffold is thus not necessarily identical in sequence length to the sequence that is used to replace the endogenous miRNA sequence. The artificial miRNA that is produced from the miRNA scaffold also not necessarily comprise the exact sequence that is used to replace the wild-type miRNA sequence. Thus, the miRNA sequence comprises or consists of the first RNA sequence, or the miRNA sequence comprises in whole or a substantial part of the first RNA sequence, said miRNA sequence being capable of sequence specifically targeting a gene, e.g. a gene transcript. Hence, as long as the miRNA produced from the miRNA scaffold is capable of inducing RNAi, such a scaffold is contemplated in the invention. The artificial miRNA may thus preferably be comprised in a pre-miRNA scaffold or a pri-miRNA scaffold.

As shown in the examples, an artificial miRNA may be preferably incorporated in a pre-miRNA or a pri-miRNA scaffold derived from microRNA451a. The terms ‘microRNA451a’, ‘miR451’, ‘451 scaffold’ or simply ‘451’ are used interchangeably throughout this specification. This scaffold allows to induce RNA interference resulting in only guide strand induced RNA interference. The pri-miR451 scaffold does not result in a passenger strand because the processing is different from the canonical miRNA processing pathway (Cheloufi et al., Nature, 2010 Jun 3;465(7298):584-9; Cifuentes et al, Science, 2010, 328 (5986), 1694-1698 and Yang et al., Proc Natl Acad Sci USA. 2010 Aug. 24; 107(34):15163-8). Hence, this scaffold represents an excellent candidate to develop a gene therapy product as unwanted potential off-targeting by passenger strands can be largely, if not completely, avoided.

As an alternative to the miR451 scaffold, similar Dicer independent structures may be preferably employed such as described herein and i.a. in Herrera-Carrillo and Berkhout, N A R, 2017, Vol. 45 No. 18 10369-79, which is incorporated herein by reference. As a passenger strand may result in off-targeting e.g. targeting transcripts other than the desired target, using such a scaffold may allow one to avoid such unwanted targeting.

As shown in the examples, a first RNA sequence of 22 nucleotides (e.g. for a miR451) in length may be selected and incorporated in a miRNA scaffold. Such a miRNA scaffold sequence is subsequently processed by the RNAi machinery as present in the cell. When reference is made to miRNA scaffold it is understood to comprise pri-miRNA structures or pre-miRNA structures. miRNA scaffolds based on 451, when processed in a neuronal cell, can result in guide sequences, i.e. an artificial miRNA, comprising the first RNA sequence (the sequence that replaced the endogenous 451 miRNA sequence) or a substantial part thereof, having a length which is in the range of 19-30 nucleotides as shown in the examples. Such guide strands being capable of reducing the target gene expression by targeting the selected target sequences. As is clear from the above, the first RNA sequence as it is encoded by the expression cassette of the invention, is comprised in part or in whole, in a guide strand when it has been processed by the RNAi machinery of the cell. Hence, the guide strand, i.e. artificial miRNA, that is to be generated from the RNA encoded by the expression cassette, comprising the first RNA sequence and the second RNA sequence is to comprise at least 18 nucleotides of the first RNA sequence. Preferably, such a guide strand comprises at least 19 nucleotides, 20 nucleotides, 21 nucleotides, or at least 22 nucleotides. A guide strand can comprise the first RNA sequence also as a whole. In selecting a miRNA scaffold e.g. from miRNA scaffolds as found in nature such as in humans, the first RNA sequence can be selected such that it is to replace the original guide strand. This does not necessarily mean that a guide strand produced from such an artificial scaffold are identical in length and sequence to the first RNA sequence selected, nor may it necessarily be so that the first RNA sequence is in its entirety to be found in the guide strand that is produced.

A miRNA451 scaffold, preferably comprises from 5′ to 3′, firstly 5′-CUUGGGAAUGGCAAGG-3′ (SEQ ID NO. 20), followed by a sequence of 22 nucleotides, comprising or consisting of the first RNA sequence, followed by a sequence of 17 nucleotides, which is complementary over its entire length with nucleotides 2-18 of said sequence of 22 nucleotides, subsequently followed by sequence 5′-CUCUUGCUAUACCCAGA-3′ (SEQ ID NO. 21). Preferably the first 5′-C nucleotide of the latter sequence is not to base pair with the first nucleotide of the first RNA sequence. Such a scaffold may comprise further flanking sequences as found in the original pri-miR451 scaffold. Alternatively, the flanking sequences, may be replaced by flanking sequences of other pri-mRNA structures. It is understood that, as the miR451 scaffold can provide for guide strands only due to the length of the stem sequence, it is preferred that alternative flanking sequences do not extend the stem length of 17 consecutive base pairs. As is clear from the above, the sequence of the scaffold may differ not only with regard to the (putative) guide strand sequence, and sequence complementary thereto, as present in the wild-type scaffold, but may also comprise additional mutations in the 5′sequence, loop sequence and 3′ sequence as well, as additional mutations may be required to provide for an RNA structure that is predicted to mimic the secondary structure of the wild-type scaffold and/or does not have a stem extending beyond 17 consecutive base pairs. Such a scaffold may be comprised in a larger RNA transcript, e.g. a pol II expressed transcript, comprising e.g. a 5′ UTR and a 3′UTR and a poly A. Flanking structures may also be absent. An expression cassette in accordance with the invention thus expressing a shRNA-like structure having a sequence of 22 nucleotides, comprising or consisting of the first RNA sequence, followed by a sequence of 17 nucleotides, which is complementary over its entire length with nucleotides 2-18 of said sequence of 22 nucleotides, and further comprising 1 or more additional nucleotides which is predicted not to form a base pair with the first RNA sequence. The latter shRNA-like structure derived from the miR451 scaffold can be referred to as a pre-miRNA scaffold from miR451.

Although the invention is typically concerned with miRNA, other forms of gene therapy products, in particular other nucleic acids, may also be detected in CSF. CSF in the context of this invention means the clear, colourless body fluid found in the brain and spinal cord. It is found i.a. in the subarachnoid space (between the arachnoid mater and the pia mater) and the ventricular system around and inside the brain and spinal cord. It fills the ventricles of the brain, cisterns, and sulci, as well as the central canal of the spinal cord. It may be obtained from a subject in any manner known to the skilled person. A preferred method of obtaining a sample may be a lumbar puncture, which is a standard procedure in medical care. According to the invention CSF is the preferred source for extracellular vesicles, which is connected with the invention's preferred diseases to be treated. Neurodegenerative diseases are often proposed to be treated by gene delivery to the brain, of which the CSF is the drainage system. In other diseases other body fluids, such as blood (serum/plasma), urine or saliva may be chosen, but other body fluids may also be employed in neurodegenerative diseases.

In one embodiment, the invention relates to gene delivery in the brain combined with detection of any product expressed from the gene delivery vehicle in the CSF.

Extracellular vesicles (exosomes and microvesicles in particular) are also produced from other cells in the body and typically exosomes and/or microvesicles from these cells may end up in other body fluids. Thus there is typically a combination of target cells the gene delivery vehicle is provided to and body fluids in which the extracellular vesicles (containing the gene delivery product, in particular the miRNA) derived from the target cells will end up. The invention however is particularly useful for gene therapy whereby the gene delivery vehicle is delivered to the CNS, such as the brain, because in that case there may be few suitable (if any) other body fluids available that can be used to monitor the expression of the gene delivery product. That means that the methods of the invention are particularly preferred for methods according to the invention wherein the miRNA interferes with the expression of a nucleic acid involved in a neurodegenerative disease. Neurodegenerative diseases according to the invention include, but are not limited to Huntington's disease, amyotrophic lateral sclerosis (ALS), spinal muscular atrophy (SMA), spinocerebellar ataxias (SCAs), Parkinson's disease, and tauopathies including Alzheimer's disease (AD) and a major class of frontotemporal degeneration (FTD), such as progressive supranuclear palsy (PSP), corticobasal degeneration (CBD) and Pick's disease.

There are a number of targets that can be addressed by the gene delivery vehicle products of the invention. In principle any target that can be beneficially downregulated using interfering RNA, in particular miRNA is a suitable target according to the invention. It may be preferred to downregulate the expression of a protein that causes health issues, e.g. because it is an aberrant protein, possibly even toxic and/or may form aggregates. It may be also be preferred to downregulate the expression of an RNA transcript that causes health issues, e.g. because it is an aberrant RNA transcript, possibly even toxic and/or may form aggregates.

Thus, in particularly preferred according to the invention is a method wherein said disease to be treated is Huntington's disease, or SCA3.

As stated herein before there are many known gene delivery vehicles, both viral and non-viral. Said gene delivery vector is to comprise an expression cassette comprising the nucleic acid encoding the miRNA in accordance with the invention. Preferably, gene delivery vehicles are used that can stably transfer the nucleic acid and/or expression cassette to cells in a human patient such that expression of the artificial miRNA can be achieved. Suitable vectors may be lentiviral vectors, retrotransposon-based vector systems, or AAV vectors. It is understood that as e.g. lentiviral vectors carry an RNA genome, the RNA genome (a nucleic acid) will encode for the said expression cassette such that after transduction of a cell and reverse transcription a double stranded DNA sequence is formed comprising the nucleic acid sequence and/or said expression cassette in accordance with the invention.

All such vehicles are within the scope of the present invention, but the preferred vehicle is based on AAV vectors. AAV sequences that may be used in the present invention for the production of AAV vectors, e.g. as produced in insect or mammalian cell lines, can be derived from the genome of any AAV serotype. The production of AAV vectors comprising an expression cassette of interest is described i.a. in; WO2007/046703, WO2007/148971, WO2009/014445, WO2009/104964, WO2011/122950, WO2013/036118, which are incorporated herein in its entirety. Generally, the AAV serotypes have genomic sequences of significant homology at the amino acid and the nucleic acid levels, provide an identical set of genetic functions, produce virions, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71: 6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74: 8635-47). AAV serotypes 1, 2, 3, 4 and 5 may be preferred source of AAV nucleotide sequences for use in the context of the present invention. Preferably the AAV ITR sequences for use in the context of the present invention are derived from AAV1, AAV2, and/or AAV5. Likewise, the Rep52, Rep40, Rep78 and/or Rep68 coding sequences are preferably derived from AAV1, AAV2 and AAV5. The sequences coding for the VP1, VP2, and/or VP3 capsid proteins for use in the context of the present invention may preferably be taken from AAV1, AAV2, AAV3, AAV4, AAV5, AAV7, AAV8, AAV9, AAVrh10 and AAV10 as these are serotypes that may be suitable for use in transducing the CNS. Also, newly developed AAV-like particles obtained by e.g. capsid shuffling techniques and AAV capsid libraries comprising mutations (insertions, deletions, substitutions), derived from AAV capsid sequences, and selected from such libraries as being suitable for CNS, e.g. neuronal cell, transduction may be contemplated. AAV capsids may consist of VP1, VP2 and VP3 capsid proteins, but may also consist of VP1 and VP3 capsid proteins. AAV capsids may not contain any substantial amount of VP2 capsid protein. This is because the VP2 capsid protein may not be essential for efficient transduction.

A preferred AAV vector that may be used in accordance with the inventions is an AAV vector of serotype 5. AAV of serotype 5 (also referred to as AAV5) may be in particularly useful for transducing human neuronal cells. AAV vectors comprising AAV5 capsids can comprise AAV5 VP1, VP2 and VP3 capsid proteins. AAV vectors comprising AAV5 capsids can also comprise AAV5 VP1 and VP3 capsid proteins, while not comprising AAV5 VP2 capsid proteins or at least not comprising any substantial amount of VP2 capsid proteins. In a wild-type derived AAV5 capsid protein, the VP1, VP2 and VP3 capsid proteins comprise identical amino acid sequences at their C-termini. The VP3 sequence is comprised in the VP2 sequence, and the VP2 sequence is comprised in the VP1 sequence. The N-terminal part of the VP1 amino acid sequence that is not contained in the VP2 and VP3 capsid proteins is positioned at the interior of the virion. This N-terminal VP1 sequence may e.g. be exchanged with an N-terminal sequence of another serotype, e.g. from serotype 2, whereas the VP2 and VP3 amino acid sequences may be entirely based on the AAV5 serotype. Such non-natural capsids comprising hybrid VP1 sequences, and such hybrid vectors are also understood to be AAV5 viral vectors in accordance with the invention. Such a hybrid vector of the AAV5 serotype is i.a. described by Urabe et al., J Virol. 2006. Furthermore, AAV5 capsid sequences may also have one or more amino acids inserted or replaced to enhance manufacturing and/or potency of a vector, such as i.a. described in WO2015137802. Such modified AAV5 capsids are also understood to be also of the AAV5 serotype. AAV (also referred to as AAV vector) is preferred because it may remain episomal for a long time, thus giving prolonged expression, but having a very low integration frequency into the host genome, with a very low risk of undesired integration at undesired sites. As the preferred gene delivery vehicles are intended to treat diseases of the brain, the invention has as a preferred embodiment a method wherein said miRNA expressed in the brain is expressed through the introduction of a gene delivery vehicle in the brain. A preferred route of administration of AAV may be to the CSF, e.g. intrathecally, such as described e.g. in WO2015060722 or Watson, et al., Gene Therapy, 2006. An alternative route of administration may be intraparenchymal or subpial administration. The artificial miRNA to be delivered according to the invention is preferably comprised in a 451 scaffold. The miRNA451 scaffold has been disclosed in WO2011133889 and WO2016102664. It has as one of its advantages that is does not generate passenger strands, but more importantly, the present inventors have shown that it can be used as a scaffold to generate artificial miRNAs that are incorporated into extracellular vesicles quite efficiently, thereby making its detection therein possible and reliable.

According to the invention the artificial miRNA may be detected/quantified by method known in the art, but preferred is a method wherein the abundance of said miRNA present in extracellular vesicles is determined by biofluid extracellular vesicle-enrichment methods (e.g. size exclusion chromatography, ultracentrifugation, density-based separation, immunoaffinity capture, membrane affinity spin columns) (Mateescu et al., J Extracell Vesicles. 2017 Mar. 7; 6(1):1286095; Enderle et al., PLoS One. 2015 Aug. 28; 10(8):e0136133; Böing et al., J Extracell Vesicles. 2014 Sep. 8; 3) and followed by RNA isolation (Eldh et al., Mol Immunol. 2012 April; 50(4):278-86) and downstream RT-qPCR (using custom-made Tagman or LNA-based assays) (Chen et al., Nucleic Acids Res. 2005 Nov. 27; 33(20):e179; Androvic et al., Nucleic Acids Res. 2017 Sep. 6; 45(15):e144) or by hybridization-based direct miRNA detection methods (e.g. SMARTbase technology in combination with single molecule array assay) (Rissin et al., PLoS One. 2017 Jul. 5; 12(7):e0179669).

In a further preferred embodiment the abundance of the miRNA is correlated with the progression or the inhibition thereof of the disease. This may be achieved by measuring both the abundance of the relevant miRNAs and the presence and/or abundance of at least one disease marker. In a particularly preferred embodiment the disease is Hungtington and the abundance of miRNA in CSF and/or serum/plasma is correlated with at least one of N-acetyl aspartate levels and/or myoinositol levels in target regions of the brain, or with mutant huntingtin protein in the CSF, or with neurofilament light chain (NFL) in the CSF or in serum/plasma.

The detection of N-acetyl aspartate levels and/or myoinositol levels in target regions of the brain is described for relevant Huntington disease preclinical models (Zacharoff et al., J Cereb Blood Flow Metab. 2012 March;32(3):502-14; Heikkinen et al, PLoS One. 2012;7(12):e50717; Peng et al., PLoS One. 2016 Feb. 9; 11(2):e0148839) and Huntington disease patients (Sturrock et al., Neurology. 2010 Nov. 9; 75(19):1702-10; van den Bogaard et al., J Neurol. 2011 December; 258(12):2230-9; van den Bogaard et al., J Huntingtons Dis. 2014; 3(4):377-86; Sturrock et al., Mov Disord. 2015 March;30(3):393-401) and can be carried out using magnetic resonance imaging scanners of different strengths (starting from 1.5 Tesla and above) and adequate scanning sequences to perform proton magnetic resonance spectroscopy (1H MRS) in target regions (including but not limited to striatum and cerebral cortex).

Preferred scanning sequences to perform 1H MRS in the clinical setting include but are not limited to sequences provided in commercial MRS packages such as stimulated echo acquisition mode (STEAM) (Frahm et al., J Magn Reson 1987; 72:502-508) and point resolved spectroscopy (PRESS) (Bottomley et al., Annal NY Acad Sci 1987; 508:333-348) and other highly optimized pulse sequences such as STEAM, SPECIAL and semi-LASER (sLASER) (for an overview see Deelchand et al., Magn Reson Med 2018 Mar;79(3):1241-1250).

The detection of mutant huntingtin protein in the CSF is described in Wild et al. (J Clin Invest. 2015 May; 125(5):1979-86) and Fodale et al. (J Huntingtons Dis. 2017; 6(4):349-361). The assay is based on the use of a capture antibody, coated on magnetic beads, and a detection antibody labelled with a fluorescent dye. The analyte is quantified with respect to standard curve of recombinant huntingtin protein (N548 Q72 HTT). The following antibodies, but not limited to these, can be used for the assay: 2B7 antibody (licenced from Novartis) used as capture and directed against the 17 N-terminal amino acids of the huntingtin protein; MW1 (licenced from Caltech) antibody, used as detection, which binds to the expanded poly-glutamine domain of mutant huntingtin protein. A high sensitivity immunoassay platform is required for the detection of mutant huntingtin protein in CSF.

The detection of NFL in the CSF and serum/plasma is described in Byrne et al. (Lancet Neurol. 2017 Aug;16(8):601-609) and can be carried out using antibody-based immunoassay with specific antibodies available from Uman Diagnostics (umandiagnostics.com/nf-light-product/nf-light-reagents/), using standard protocols. For detection of NFL in CSF, a wide range of immunoassay platforms can be used, while for the detection of NFL in serum/plasma, a high sensitivity immunoassay platform is required.

Preferred detection methods (immunoassay platforms) for mutant huntingtin protein in CSF and NFL in CSF or serum/plasma are antibody-based detection methods, using including but not limited to standard platforms like ELISA, MSD and Luminex, and high sensitivity platforms such as Singulex and SIMOA.

The invention will be explained in more detail in the following experimental section.

### EXAMPLES

### Example 1

### Material and Methods

Expression Cassettes, miRNAs and AAV Vectors

Expression cassettes and AAV vectors used in the studies are as described i.a. in WO2016102664 and Miniarikova et al., 2016. The expression cassette was inserted into a vector genome backbone flanked by two intact non-coding inverted terminal repeats (ITR) that originate from AAV2. Briefly, miRNA expression cassettes comprise the chimeric chicken-beta actin promoter, the miRNA sequence was replaced by a sequence designed to target a selected gene sequence and engineered in the pri-mir-451 backbone, and the human growth hormone polyA signal (Schematically depicted in FIGS. 1A-1B). The sequence of an exemplary expression cassette is depicted in FIG. 2. The 22 nucleotide sequence targeting ATXN-3 was 5′-TCTGGAACTACCTTGCATACTT-3′ (SEQ ID NO. 2; 22 nts) and is comprised in the sequence depicted in FIG. 2. The sequence of an exemplary expression cassette is depicted in FIG. 2. The 22 nucleotide sequence targeting the Huntington gene sequence that was used in these experiments corresponds with 5′-AAGGACTTGAGGGACTCGAAGA-3′ (SEQ ID NO. 3; 22 nts).

The sequence targeting the Huntington gene sequence corresponds with the H12 candidate as described in WO2016102664 and Miniarikova et al., 2016, which is incorporated herein by reference. The sequences selected targeting the ATXN3 and HTT genes represent sequences that, when expressed, and processed by the RNAi machinery, are complementary to target sequences in mRNAs expressed from the ATXN3 and HTT genes, respectively. Hence, the RNA sequences that are complementary to HTT and ATNX3, respectively, when comprised in a miRNA scaffold as depicted e.g. in FIGS. 1A-1B, correspond respectively with 5′-AAGGACUUGAGGGACUCGAAGA-3′ (SEQ ID NO. 5; 22 nts), the sequence that was used to target ATXN-3 was 5′-UCUGGAACUACCUUGCAUACUU-3′ (SEQ ID NO. 6; 22 nts). AAV vectors used in these studies were based on the AAV5 serotype and manufactured using insect cell based manufacturing. Briefly, Recombinant AAV5 harbouring the expression cassettes were produced by infecting SF+ insect cells (Protein Sciences Corporation, Meriden, Connecticut, USA) as described (Lubelski et al. Bioprocessing Journal, 2015). Following standard protein purification procedures on a fast protein liquid chromatography system (AKTA Explorer, GE Healthcare, Chicago, Illinois, USA) using AVB sepharose (GE Healthcare, Chicaco, Illinois, USA), the titer of the purified AAV was determined using qPCR.

Small RNAseq analysis of the mature miRNA sequences produced from the 451 scaffold targeting the Huntington gene (in order of abundance, expressed as %) are listed below, (miHTT). The sequences listed correspond with RNA sequences, i.e. the sequences listed below are DNA sequences, which, when the Ts are replaced with Us, represents the corresponding RNA sequences.

| SEQ ID | | | % |
|---|---|---|---|
| NO. | SEQUENCE (5′-NNN-3′) | length | reads |
| 7 | AAGGACTTGAGGGACTCGAAGACG | 24 | 45.7 |
| 8 | AAGGACTTGAGGGACTCGAAGACGAGT | 30 | 16.0 |
| | CCC | | |
| 9 | AAGGACTTGAGGGACTCGAAGAC | 23 | 11.5 |
| 3 | AAGGACTTGAGGGACTCGAAGA | 22 | 8.3 |
| 10 | AAGGACTTGAGGGACTCGAAGACGA | 25 | 4.9 |
| 11 | AAGGACTTGAGGGACTCGAAG | 21 | 3.1 |
| 12 | AAGGACTTGAGGGACTCGAAGACGAGT | 27 | 2.3 |
| 13 | AAGGACTTGAGGGACTCGA | 19 | 1.6 |
| 14 | AAGGACTTGAGGGACTCGAAGACGAGT | 31 | 1.1 |
| | CCCT | | |
| 15 | AAGGACTTGAGGGACTCGAA | 20 | 1.0 |

Small RNAseq analysis of the mature miRNA sequences produced from the 451 scaffold targeting the ATXN3 gene (in order of abundance, expressed as %) are listed below (miATXN). The sequences listed correspond with RNA sequences, i.e. the sequences listed below are DNA sequences, which, when the Ts are replaced with Us, represents the corresponding RNA sequences.

| SEQ ID | | | % |
|---|---|---|---|
| NO. | SEQUENCE (5′-NNN-3′) | length | reads |
| 2 | TCTGGAACTACCTTGCATACTT | 22 | 53.0 |
| 16 | TCTGGAACTACCTTGCATACTTAT | 24 | 18.0 |
| 17 | TCTGGAACTACCTTGCATACTTA | 23 | 11.0 |
| 18 | TCTGGAACTACCTTGCATACT | 21 | 6.1 |
| 19 | TCTGGAACTACCTTGCATACTTAT | 30 | 2.9 |
| | GCAAGG | | |

The most abundant sequences were used for the analysis of CSF and EV, i.e. the 24 nts sequence corresponding SEQ ID NO.7 was used in the analysis representing miRNA found in CSF or EV, likewise, the sequence corresponding with SEQ ID NO.2 was used in the analysis representing miRNA found in EV. For analysis of brain samples, for both ATXN3 and HTT the 22 nucleotide sequences were used in the analysis.

Differentiation of Forebrain Neuronal Cultures from Human HD Induced Pluripotent Stem Cells (iPSCs)

HD iPSCs (ND42229*B) containing 71 CAG repeats were ordered from (Coriell Institute Stem Biobank, passage 25). These cells were generated from human HD fibroblasts GM04281 (Coriell Institute Stem Biobank) reprogrammed with six factors (OCT4, SOX2, KLF4, LMYC, LIN28, shRNA to P53) using episomal vectors. iPSCs were maintained on matrigel coating with mTeSR medium for several passages, following the manufacturer's instructions (StemCell Technologies, Vancouver, Canada). Non-differentiated colonies were released using ReLeSR reagent during each passage and diluted 1:5-20 (StemCell Technologies). Forthe neural induction, cells were plated onto AggreWell™ 800 plate at day 0 as 3×106 cells per well in STEMdiff™ Neural Induction Medium (StemCell Technologies). At day 5, embryoid bodies were formed and replated onto poly-D-lysine/laminin coated 6-well plates. Coating was prepared with poly-D-lysine hydrobromide (0.1 mg/mL) and Laminin from Engelbreth-Holm-Swarm murine (0.1 mg/ml) (Sigma-Aldrich). At day 12, the neuronal rosettes were selected using STEMdiff™ Neural Rosette Selection Reagent (StemCell Technologies) and replated in poly-D-lysine/laminin coated plates. Next day, differentiation of neural progenitor cells was initiated using STEMdiff™ Neuron Differentiation Kit (StemCell Technologies). From day 19, cells were matured using STEMdiff™ Neuron Maturation Kit for a minimum of two weeks (StemCell Technologies).

Extracellular Vesicles (EVs) Precipitation and Detection of miRNA Secreted by iPSC-Derived Neurons

Cells were transduced with different doses of AA5-miHTT (3E11gc, 3E12gc and 3E13gc) or AAV5-miATXN (3E12gc and 3E13gc). Medium from transduced neuronal cultures was refreshed every two days and collected on day 5 and day 12 after transduction, and centrifuged at 3000×g for 15 min to remove cells and cell debris. The EVs were isolated with ExoQuick-TC (System Bioscience, California, USA) according to manufacturer's protocol. 3 ml of ExoQuick buffer was added to 10 ml of conditioned medium and incubated at 4° C. overnight. Next day, the exosomes were collected at 1500×g for 30 minutes and the supernatant was discarded. The residual solution was additionally centrifuged at 1500×g for 10 minutes. The EVs pellets were re-suspended in appropriate buffers and stored at −80° C. for further experiments.

RNA Isolation and Real-Time qPCR

RNA was isolated from cells using Trizol according to the manufacturer's protocol (Invitrogen). To measure mRNA knock-down, cDNA was generated using Maxima Synthesis Kit (Thermo Scientific). cDNA was analyzed by qPCR using commercially available primers and probes: human HTT (assay ID Hs00918134 ml) and human GAPDH (assay ID Hs02758991_g1) (corresponding primer and probes available using assay IDs at Thermo Scientific). To detect miHTT expression levels, RT-qPCR was performed using TaqMan Fast Universal kit (Thermo Scientific), and commercially available primers and probes hsa-miR-16 (000391, Applied Biosystems). Custom-made miHTT primers and probes (assay ID CTXGPY4, Thermo Scientific) and miATXN primers and probes (assay ID CTEPRZE, Thermo Scientific), were used for SEQ ID NOs. 7 and 2, respectively. The expression level was normalized to hsa-miR-16 levels. Fold changes of miRNA expression were calculated based on 2\hat{ }ΔΔCT method (Livak and Schmittgen, Method. Methods. 2001, Dec;25(4):402-8). miRNA expression was calculated based on a standard line with synthetic RNA oligos. For the viral vector DNA isolation, neuronal cultures were processed using DNeasy Blood & Tissue Kit (Qiagen, Valencia, CA, USA) following manufacturer's protocol. AAV5 vector genome copies were measured by qPCR reaction using SYBR Green protocol (Applied Biosystems, Foster City, CA, USA) and validated standard line for detection of CAG promoter. Forward primer sequence: GAGCCGCAGCCATTGC and reverse primer sequence: CACAGATTTGGGACAAAGGAAGT (SEQ ID NO.23-24). The standard line was used to calculate the genome copies per DNA microgram.

### Protein Isolation and Western Blotting

EVs precipitates were lysed using RIPA lysis buffer (Sigma-Aldrich) supplemented with protein inhibitor cocktail (cOmplete™ ULTRA Tablet; Roche, Basel, Switzerland). Total protein concentration was quantified using a Bradford Protein Assay (Bio-Rad, Hercules, CA, USA) and absorbance was measured at 600 nm on the GloMax Discover System (Promega). Equal amounts of sample protein (10-g) were incubated with β-mercaptoethanol and Laemmli buffer at 95° C. for 5 min and separated using 4-20% Mini-Protean TGX Stain-Free Protein Gel (Bio-Rad). Samples were transferred to PVDF membranes by Trans-Blot Turbo Transfer system (Bio-Rad) using the “Mixed MW” protocol. Blot was incubated with 3% Blotting-Grade blocker (Bio-Rad) in 1× Tris Buffered Saline (TBS) for 1 hour at room temperature, followed by immunoblotting with selected primary antibody overnight at 4° C. (see Table 1 below). Chromogenic signals were detected after 2 hours incubation with HRP-conjugated secondary antibodies (see table below) and 5 min incubation with SuperSignal Pico sensitivity Substrate (ThermoScientific) using ChemiDoc Touch Gel Imaging System (Bio-Rad).

TABLE 1
Antibodies used for Western Blotting

| | Antibodies | Brand | Ordering number | Concentration |
|---|---|---|---|---|
| EVs | CD63 | System Biosciences | EXOAB- | 1:1000 |
| | Alix | Abcam | CD63A-1 | 1:1000 |
| | TSG101 | Abcam | ab76608 | 1:1000 |
| | | | ab30871 | |
| ER | Calnexin | Abcam | ab92573 | 1:20000 |
| Mitochondria | ATPase | Abcam | ab58475 | 1:1000 |
| RISC | Ago2 | EMD Millipore | 07-590-25UG | 1:1000 |
| Cytoplasm | a-tubulin | Abcam | ab7291 | 1:1000 |
| | [DM1A] | | | |
| Secondary | HRP goat anti- | Abcam | ab97051 | 1:20000 |
| antibodies | rabbit | | | |
| | HRP rabbit anti- | DAKO | | 1:20000 |
| | mouse | | | |

### Results

Detection of miRNAs in the Medium from AAV-Transduced iPSC-Derived Neurons after 5 Ad 12 Days.

HD iPSC-derived neuronal cells were transduced with two therapeutic viral vectors: AAV5-miHTT and AAV5-miATXN. Results show a dose-dependent transduction of HD-iPSC derived neuronal cells with three doses of AAV5-miHTT (3E11gc, 3E12gc and 3E13gc) and two doses of AAV5-miATXN (3E12gc and 3E13gc) at 20 days after transduction (FIG. 3). After 5 and 12 days, both miHTT and miATXN molecules were detected in a dose-dependent manner in the EVs precipitated from the medium of neuronal cells. Graphs depicted in FIGS. 4 and 5 represent fold change expression of miHTT and miATXN, respectively, normalized to endogenous miR-16 and compared to control cells.

EV-miHTT Secreted from Transduced Cells Strongly Correlates with AAV Dose and miHTT Expression in the Cells

We summarized data from independent experiments showing a robust correlation between secreted miHTT molecules detected in the medium of transduced cells and both viral vector dose (FIG. 6A) and miHTT expression within the cells (FIG. 6B) (n=6). Experiments were performed separately at different time points. Graphs represent AAV dose (log 10) and relative expression of miHTT detected by qPCR. Each dot represents the average of an independent experiment which was performed in duplicate or triplicate.

Characterization of EVs Precipitated from Medium of iPS-Derived Neurons by Western Blot

In order to confirm the composition of EVs precipitated from the medium, protein was isolated and analyzed by western blot with different markers (See Methods section) (FIG. 7). Results confirmed a presence of EV and exosomal markers (CD63, Alix and TSG-101), microvesicles (Calnexin) and RISC complex (Ago2) to which functional miRNAs are bound to. Cellular markers (a-tubulin and ATPase) were used as controls to confirm the absence of cells or cellular debris.

Therapeutic miRNA Molecules Secreted from AAV5-Transduced Neuronal Cells can be Transferred to Naïve Neuronal Cultures in a Dose-Dependent Manner.

Medium from PBS, AAV5-miHTT and AAV5-miATXN transduced iPSC-derived neuronal cells was collected and EVs precipitated by Exoquick (see Methods section). EVs pellets were pooled together and added in different concentrations (0.1×, 0.5× 1×, 2× or 5×) to 1E5 naive iPSC-derived neuronal cells in one well of a 24-well plate (see FIG. 8A). Cells were harvested 24 hours after EV-transfer. Results depicted in FIGS. 8B and 8C indicate a dose-dependent transfer of miHTT and miATXN to naïve neuronal cells.

Non-Viral Vector Functional Transfer of Therapeutic miRNAs Between Neuronal Cells.

iPS-derived neuronal cells transduced with 3E13 gc AAV5-miHTT from the previous experiment were seeded in Corning® Transwell® polyester membrane cell culture inserts (24 mm, 0.4 m pore) (Sigma). Naïve iPS-derived neurons were seeded 5E5 neurons/well in a 6-well plate. 48 hours after seeding, inserts were placed on top of the wells and cells were cocultured for 2 weeks, refreshing medium every 2 or 3 days (n=6). Naïve iPSC-derived cells without inserts were used as controls. Cells were harvested separately with Accutase (STEMcell technologies) and divided for different molecular analysis including AAV5 genome copies (FIG. 9A) and HTT mRNA lowering (FIG. 9B) by real-time qPCR (see Methods section). Results show that there was no viral vector genome transfer between donor and recipient cells since only donor cells in Transwell inserts contained high levels of AAV5 genome copies detected by qPCR (n=6). Interestingly, in both donor transduced cells and recipient naïve cells we detected a 30% and 20% HTT mRNA lowering, respectively, compared to control cells (n=6). This preliminary data indicates a functional transfer of therapeutic miRNAs between neuronal cells.

### Conclusions

To conclude, therapeutic miRNA molecules are present in a dose-dependent manner in the EVs precipitated from the supernatant of HD neuronal cultures treated with AAV5-miHTT-451 and AAV5-miATXN-451 gene therapies. Extracellular therapeutic miRNAs levels secreted from transduced cells strongly correlate with AAV5 dose and miRNA expression in the cells. Therapeutic miRNA molecules within EVs secreted from AAV5-transduced neuronal cells can be transferred to naïve HD neuronal cultures in a dose-dependent manner. Combined this data indicated viral vector expressed therapeutic miRNAs can be functionally transferred between neuronal cells.

### Example 2

This example assessed miHTT-24nt microRNA expression targeting mutant HTT gene in CSF extracellular vesicles from non-human primates (cynomolgus monkeys).

### Study Design

This study in male and female cynomolgus monkeys employs a gene therapy product designed to silence the expression of huntingtin by means of the RNA interference (RNAi) mechanism using an engineered microRNA (miHTT) targeting human huntingtin, delivered via adeno-associated viral vector serotype 5 (AAV5-miHTT) in the brain. The design of the study is outlined in Table 2 below.

TABLE 2
Study design

| | | Dose level (gc/brain) and | | Number | |
|---|---|---|---|---|---|
| | | volume (in uL per target region) | | of animals | |
| Group | Treatment | Dose level | Volume | Male | Female |
| 1 | Control | 0 (PBS) | 4 × 100 | 3 | 3 |
| 2 | Low | 2E12 | 4 × 100 | 3 | 3 |
| 3 | Mid | 7E12 | 4 × 100 | 3 | 3 |
| 4 | High | 2E13 | 4 × 100 | 3 | 3 |

Animals were injected with AAV5-miHTT at different doses as indicated in the table. AAV5-miHTT was delivered bilaterally in the brain, directly in the caudate and putamen (100 uL per region), by MRI-guided convention-enhanced delivery (CED). From each animal, CSF was collected at pre-dose and at 4/5, 13/14, 18/H19 and 24/15 weeks post-dosing. Only samples from AAV5-miHTT dosed groups (2-4) were analyzed for miHTT levels in extracellular vesicles.

### Materials and Methods

The materials and equipment used during the performed experiments are listed below

Materials used during the experiments

| Materials | Company | Lot nr |
|---|---|---|
| exoRNeasy Serum/Plasma midi kit | QIAGEN | 160025144 |
| QIAzol® Lysis Reagent | QIAGEN | 557015613 |
| Chloroform | EMPLURA® | K46454865515 |
| Ethanol (100%) | EMPLURA® | K50144683815 |
| miRNeasy Serum/Plasma Spike-In control | Qiagen | 160020299 |
| 24 nt_guide_miHTT (24 bases) SEQ ID NO. 7 | Integrated | Ref: 75930597 |
| (5′AAGGACUUGAGGGACUCGAAGACG3′) | DNA | |
| RNase-free 1.5 ml microfuge tubes | Technologies | 09117018 |
| | Ambion | |
| RNase-free 8-strip 0.2 ml PCR tubes w/caps | Invitrogen | 143A7 |
| RT primer Celmir39 | IDT | Assay 00020 |
| RT primer miHTT24 | IDT | Assay CTXGPY4 |
| Taqman microRNA Reverse transcription kit | Applied | 00623180 |
| | Biosystems | |
| DEPC Nuclease free water | Ambion | 1709015 |
| TM primer probe Celmir39 | Applied | Assay lot#00020 |
| | Biosystems | |
| TM primer probe miHTT24 | Applied | Assay lot#CTXGPY4 |
| | Biosystems | |
| Taqman Fast Universal PCR master mix (2x) | Applied | 17104895 |
| | Biosystems | |

### RNA Isolation

CSF samples were thawed at 4° C., shortly spun down at 4° C., and 450 ul CSF (or the maximum volume available) transferred to a new 2 mL tube. When 450 ul CSF where not available, the maximum available volume was taken and noted (range from 360 ul to 450 ul), so that appropriate corrections per input volume could be made. Extracellular vesicle RNA was isolated using the exoRNeasy Serum/Plasma midi kit (Qiagen) following the manufacturer's instructions. As control of RNA isolation efficiency, 3.5 uL of cel-miR-39 (miRNeasy Serum/Plasma Spike-In control working solution, 1.67*108 gc/ul, prepared following the manufacturer's instructions) was added per sample. Isolated RNA was eluted in 16 ul water (DEPC-treated, RNAse free water) and stored at −80° C. until further use.

### RT-QPCR

The RT and qPCR protocols were performed in accordance with standard protocols. Briefly, for the RT reaction, 4 uL of RNA sample were added to 7 uL of RT PCR mix, and incubated in a Thermocycler according to procedures known in the art. In parallel with te RNA samples, a standard line for miHTT-24nt comprised 7 standards, ranging from 100 picogram (pg) to attogram (ag) final input miHTT-24nt RNA oligo was included in the RT reaction. The resulting cDNA samples were diluted to a final volume of 35ul each by addition of 24 ul of DEPC.

For the qPCR, 4 μL of cDNA was added to 6 μL of qPCR mix, and the qPCR reaction (40 cycles) run in the 7500 Fast Real-Time PCR (Applied Biosystems). Study samples were tested in triplicate, standards and control samples in duplicate. Amplification plots were analyzed using a fixed threshold of 0.3, and Ct values were exported to Excel for further calculations.

### RT-qPCR Controls

To exclude any reagent contamination, negative control samples were taken along within RT PCR and qPCR reactions. Additionally, a positive control (calibrator) sample was taken in each qPCR plate, to evaluate plate-to-plate comparability.

Data Processing, in-Study Validation and Acceptance Criteria

Ct values of study samples and all control samples were obtained after each QPCR run with ABI 7500 Prism software, using a fixed threshold of 0.3 for all runs. The concentrations of miHTT-24nt in the CSF study samples were derived from the miHTT-24nt RNA oligo standard line. The average result of the triplicate measurements was ultimately reported as miHTT-24nt copies per mL of CSF.

### Results

### In-Study Validation

The quality of the analysis was evaluated considering standard curve performance and quality of the positive and negative control samples. Back-calculated standard concentrations for miHTT-24nt resulted within ±20% RE % in within-run and inter-run analyses, for at least 5 of the 7 points of the standard curve. All control samples in all runs were within specifications. All analytical runs were therefore with specifications, based on the in-study validation.

### Study Results

An overview of the miHTT-24nt copies/mL CSF in extracellular vesicles is presented in the table below and FIG. 10.

| | | | | | |
|---|---|---|---|---|---|
| CSF miHTT-24nt | | | | | |
| (copies/mL) | Time- | | | | |
| Group, gender, | points | | | | |
| animal | Pre-dose | wk 4/5 | wk 12/13 | wk 18/19 | wk 25/26 |

Group 2
F

| | | | | | |
|---|---|---|---|---|---|
| P0501 | <LLoD | 2.26E+02 | 9.83E+02 | 3.79E+02 | 4.03E+02 |
| P0502 | <LLoD | 9.41E+03 | 5.09E+03 | 4.50E+03 | 4.85E+03 |
| P0503 | <LLoD | 9.20E−F03 | 1.52E+03 | 2.61E+04 | 3.87E+03 |

M

| | | | | | |
|---|---|---|---|---|---|
| P0101 | <LLoD | 1.11E+04 | 5.25E+04 | 2.92E+04 | 6.74E+03 |
| P0102 | <LLoD | 5.74E+02 | 1.85E+02 | 9.17E+02 | 3.80E+02 |
| P0103 | <LLoD | 1.46E+04 | 2.51E+04 | 4.44E+03 | 4.30E+03 |

Group 3
F

| | | | | | |
|---|---|---|---|---|---|
| P0601 | <LLoD | 1.33E+03 | 1.08E+03 | 2.87E+03 | 9.91E+02 |
| P0602 | <LLoD | 6.85E+03 | 8.02E+04 | 1.08E+04 | 3.32E+03 |
| P0603 | <LLoD | 4.39E+02 | 5.36E+01 | 8.12E+02 | 9.32E+01 |

M

| | | | | | |
|---|---|---|---|---|---|
| P0202 | <LLoD | 1.85E+03 | 2.75E+04 | 1.92E+03 | 6.97E+02 |
| P0203 | <LLoD | 3.60E+03 | 6.62E+03 | 1.76E+04 | 1.52E+03 |

Group 4
F

| | | | | | |
|---|---|---|---|---|---|
| P0701 | <LLoD | 2.24E+03 | 4.66E+03 | 7.17E+04 | 6.54E+03 |
| P0702 | <LLoD | 1.12E+04 | 3.54E+05 | 6.20E+04 | 2.30E+04 |

M

| | | | | | |
|---|---|---|---|---|---|
| P0301 | <LLoD | 4.33E+01 | 1.66E+02 | 1.33E+02 | 2.01E+02 |
| P0303 | <LLoD | 5.15E+03 | 9.58E+02 | 8.89E+02 | 3.07E+02 |

All study-samples were measured in valid runs and the results were, based on in-study validation, considered acceptable and reliable. The reported results are therefore considered representative of the concentration of miHTT-24 nt in CSF extracellular vesicles after AAV5-miHTT intra-striatal delivery in cynomolgus monkeys.

CSF samples for extracellular vesicle miHTT-24nt RT-QPCR analysis were successfully analyzed at pre-dose and at 4/5, 13/14, 18/19 and 24/25 weeks post dosing. Group mean CSF-miHTT-24nt levels were relatively constant during the 6-month observation period, around 1E4 with a trend to higher expression at 3 months with some decline thereafter to a level at 6 months comparable to the level at 4 weeks post-injection.

### Example 3

### Imaging Biomarker Study in FDNQI75 Mice

The objective of this study was to evaluate Magnetic Resonance Spectroscopy (MRS) as an exploratory longitudinal measurement of the effects of HTT lowering on neuronal metabolism in HD patients and animal models.

Clinical MRS studies in HD patients have identified a robust neurochemical signature with decreased neuronal metabolites (N-acetyl aspartate [NAA]) and elevated glial metabolites (myoinositol [MI]) in the putamen and frontal cortex of early HD compared to healthy volunteers (Sturrock et al., 2010; Sturrock et al., 2015). Similarly, the striatal neurochemical profile in the Q175FDN mouse model of HD, which expresses full-length mutant HTT protein and manifests with early motor deficits (Southwell et al., 2016), also reflects the striatal biochemical changes (decreased NAA and elevated MI) previously observed using MRS in HD subjects (unpublished observations).

Adult (12-week-old) male and female homozygous Q175FDN HD mice (genders balanced across groups) were injected bilaterally in the striatum with formulation buffer, low (5.2×10E9 gc/mouse) or high (1.3×10E11 gc/mouse) doses of AAV5-miHTT. As controls, age and gender-matched wild-type mice were used, injected with formulation buffer only. Three months after bilateral striatal injection, T1 weighted structural MRI and striatal MRS was carried out (7T animal MRI scanner) on the anesthetized mice, allowing to normalized MRS signal for brain volume loss. Animals were sacrificed directly after the MRI/MRS session, and brain tissue collected to allow for correlations between the MRS signal and biochemical changes in brain tissue (biochemical analysis ongoing).

Interim data of the MRS analysis is shown in FIGS. 11A-11B. Decreased total NAA (tNAA) and increased MI levels were found in the homozygous Q175FDN untreated mice with respect to the wild-type control animals, similar to the findings in human HD patients. Remarkably, in the Q75FDN AAV5-MIHTT treated animals this effect was reversed: significantly higher levels of tNAA and significantly reduced levels of MI were observed in the high-dose group (1.3×10E11 gc/mouse) with respect to the Q175 untreated animals.

The interim MRS analysis shows full restoration of neuronal function and partial reversal of gliosis in Q175FDN HD mice three months after AAV5-miHTT treatment and validates its potential in the clinical setting for efficacy testing in HD patients.

### Example 4

### Long-Term Efficacy Study in Transgenic Minipigs.

Long-term efficacy of AAV5-miHTT treatment is being studied in an ongoing study in 16 transgenic minipigs over several years. TgHD minipigs (male and female) were injected with a dose of 1.2×10¹³ gc/animal of AAV5-miHTT, bilaterally into the caudate nucleus and putamen (100 μL per structure) using real-time MRI-guided CED. Three minipigs of the study were sacrificed at 6 months post-injection, as well as three untreated age-matched controls. A robust reduction in human mutant huntingtin mRNA and protein was observed in the caudate nucleus, putamen, thalamus, and also, although to a lesser extent, in the frontal cortex (Vallès et al., poster 105, EHDN Plenary Meeting, Sep. 14-16, 2018, Vienna, Austria and Vallès et al. poster P187 XXVI^{th} ESGCT Congress, Oct. 16-19, 2018, Lausanne, Switzerland). In an effort to assess huntingtin protein lowering efficacy translationally, human mutant huntingtin protein was measured in longitudinal CSF samples of tgHD minipigs (n=6) before and after AAV5-miHTT treatment (Vallbs et al., poster 105, EHDN Plenary Meeting, Sep. 14-16, 2018, Vienna, Austria and Vallès et al. poster P187 XXVI^{th} ESGCT Congress, Oct. 16-19, 2018, Lausanne, Switzerland). For most of the AAV5-miHTT treated animals, there was a gradual lowering of CSF mutant huntingtin protein over time, with the strongest lowering observed at 6 months post-injection and ranging between 15% to 70% lowering, while CSF mutant huntingtin protein levels remained stable in control animals. This data confirms the efficacy of AAV5-miHTT in silencing human huntingtin in a large disease model brain, and the potential of CSF mutant huntingtin protein as a translational measure to evaluate brain huntingtin lowering.

Additional data will be obtained from this ongoing study, where CSF is being collected from AAV5-miHTT treated animals that are remaining in the study (same doses and route of administration, 1.2×10¹³ gc/animal). Animals will be sacrificed 1 year (n=4 treated and n=4 control animals) and >2 years (n=8 treated and n=8 control animals) after intraparenchymal injection to assess the persistence of the huntingtin lowering and to correlate brain and CSF HTT protein levels. Extracellular vesicle miHTT expression will also be measured in the CSF of these animals. With this information, a model predicting brain huntingtin protein lowering based on CSF miHTT and huntingtin protein determinations can be built.

### Example 5

miHTT can be Detected in tgHD Minipig Cerebrospinal Fluid (CSF) Up to 21 Months after Intrastriatal AAV5-miHTT Administration

In this study, miHTT microRNA expression was measured in CSF of tgHD minipigs every 3 months and up to 21 months post-AAV5-miHTT intraparenchymal injection in caudate and putamen.

### Animals

All experiments were carried out according to the guidelines for the care and use of experimental animals and approved by the State Veterinary Administration of the Czech Republic. TgHD minipigs (Baxa et al., J Huntingtons Dis. 2013; 2(1):47-68) and healthy controls of both sexes, 6 months old, were used.

### Study Design and Csf Collection

The whole study consisted of n=15 six months old AAV5-miHTT treated and n=15 naïve (untreated) tgHD minipigs, sacrificed at different interim timepoints (6, 12 and >2 years post-treatment). Treated animals were injected with a dose of 1.2×1013 gc/brain (bilateral injection, in caudate and putamen). At this stage, the interim sacrifices of 6 and 12 months have been performed, and the animals planned for sacrifice beyond 2 years post-treatment are still in the in-life phase. During the in-life phase, periodical lumbar CSF collections took place under anaesthesia (every three months). Collected CSF was centrifuged to remove any potential cell debris, aliquoted and stored at −80° C. until further use. For this example, CSF from different timepoints (pre-dose, 3, 6, 9, 12, 15, 18 and 21 months) from two treated animals (animal T38 and animal T45) were used.

### Materials and Methods

VECTOR—AAV5 vector encoding cDNA of the miHTT cassette was packaged into AAV5 by a baculovirus-based AAV production system (uniQure, Amsterdam, The Netherlands) as previously described (Evers et al., Mol Ther. 2018 Sep. 5; 26(9):2163-2177).

RNA ISOLATION AND RT-QPCR—RNA was isolated from 300 uL CSF, using the miRNAeasy advanced kit (Qiagen), following the manufacturer's instructions. RNA was eluted in 20 μL and stored at −80° C. Before RT-QPCR, RNA was treated with dsDNAse. To examine miHTT RNA expression, cDNA was synthesized from isolated total RNA using the miRCURY LNA miRNA PCR System (Qiagen). Next, gene-specific qPCR was performed with miHTT-specific LNA primers (Qiagen). A standard line was taken along, in order to calculate the expression of miHTT as copies/uL CSF.

### Results

In tgHD minipigs injected with AAV5-miHTT in striatum, miHTT was readily detected from 3 months up to 21 months post-injection (FIG. 13). The pattern of expression was very similar in the two animals profiled (T38 and T45), with peak levels at 3 months, decreasing and stabilizing from 6 moths on.

### Conclusions

This example demonstrated that it is feasible to detect miHTT in CSF, post-treatment with a gene therapy approach locally administered in the brain. The long-term persistence of the miHTT signal in CSF is strongly indicative of the long-term stability of the gene therapy in the brain. This supports the value of miHTT CSF as a translational measure of brain gene therapy approaches.

### Example 6

miHTT is Associated in Extravesicular and Lipoprotein Fractions from NHP CSF

In this study, the association of miHTT to extracellular vesicle and/or lipoprotein fractions obtained from cerebrospinal fluid (CSF) from non-human primates (NHPs) injected with AAV5-miHTT in caudate and putamen was determined. The miHTT association profile was compared to the profile of endogenous miRNAs known to be expressed in CSF.

### Samples

CSF samples from a study in NHP were used. In the study, adult cynomolgus monkeys were injected with AAV5-miHTT locally in caudate and putamen (dose 1-E13 gc/brain). Several longitudinal CSF samples were taken, namely on day 1 (pre-dose & post-dose 1 hr), week 2, week 4, week 8 and week 12 (prior to termination). For CSF sample collection, animals were sedated with Ketamine (10 mg/kg) and dexmedetomidine (0.015 mg/kg). Each animal's neck was shaved and its skin prepared for CSF collection. The neck was flexed and a 23 gauge needle was manually inserted in between the skull base and C1 into the cisterna magna gradually until CSF flow was established. CSF was collected upon verification of CSF flow into the needle hub and then 1.0 mL CSF sample was collected at each time point, centrifuged for 15 min at 2400 RPM, 4° C. and then divided into 2 aliquots, frozen on dry ice and stored at −70° C. until further analysis.

### Methods

SIZE EXCLUSION CHOMATOGRAPHY (SEC)—Only samples from pooled CSF of treated animals at week 2 post-injection were used for this analysis. Samples from two animals, of 500 uL each sample, were thawed in a 37° C. bath and mixed in a single Eppendorf, resulting in a final volume of 1 mL. The mix was added to a size exclusion chromatography column (custom-made) and eluted with PBS, as previously described (Böing et al., J Extracell Vesicles. 2014 Sep. 8; 3-jev.v3.23430). A total of 26 fractions of 500 uL each were collected and stored at −80° C. until further use.

RNA ISOLATIONAND RT-QPCR—A volume of 200 μL of each of the obtained SEC fractions (1-26) was used for RNA isolation using the Serum/Plasma Advanced Kit (QIAgen) and following the manufacturer's instructions. The RNA was eluted in a 16 μL of RNAse-free water. The microRNAs of interest (miHTT, miR16 and miR21) were quantified using specific Taqman assays designed for this purpose. Total RNA was used for cDNA synthesis, using TaqMan MicroRNA Reverse Transcription Kit (Applied Biosystems) together with gene-specific RT primers targeting the miRNAs of interest. For miHTT, a single stranded miHTT RNA standard line was taken along. Next, gene-specific TaqMan qPCR was performed with miHTT, miR16 or miR21-specific probes using TaqMan Fast Universal PCR Master Mix (Applied Biosystems). Using the miHTT standard line, miHTT molecules per reaction were determined, and expressed per uL of CSF used. For miR16 and miR21, the relative expression of one sample with respect to the other analyzed samples was calculated (assuming an amplification efficiency of 2).

### Results

All the analyzed miRNAs, miHTT, miR16 and miR21, were successfully detected in SEC fractions obtained from NHP CSF (two weeks post AAV5-miHTT intraparenchymal injection) (FIGS. 12A-12C). Interestingly, miHTT was associated to both vesicle (fractions 7-10) and lipoprotein (fractions 17-21) fractions. In contrast, the endogenous miRNAs miR-16 and miR-21 were primarily associated to the lipoprotein fractions (fractions 17-21).

### Conclusions

Using SEC, one of most widely accepted approaches to separate extracellular vesicles from biofluids, it was shown that miHTT is successfully detected, in both extracellular vesicle and lipoprotein fractions from NHP CSF 2 weeks after intraparenchymal injection of AAV5-miHTT. This observation supports the use of detecting miHTT in CSF as diagnostic, i.e. the detection of miHTT in CSF extracellular vesicles is a useful translational measure of brain gene therapy.

### Example 7

miATXN3 is Detected in CSF from NHP Injected with AAV5-miATXN3

In this example we show that miATXN3 can be detected, even if at low levels, in the CSF of NHP injected with AAV5-miATXN3 (combined intrathecal and intra-cisterna magna administration).

### Samples

CSF samples from a pilot study in NHP. In this pilot study, three cynomolgus monkeys were injected intrathecally (lumbar region) and intra-cisterna magna, with a total dose of 4.5 mL/animal of 5E13 gc/mL of AAV5-miATXN3. At pre-dose and 2, 4 and 8 weeks post-dosing, CSF was collected by lumbar puncture following standard procedures. CSF was briefly centrifuged to remove any possible cell contamination and stored at−80° C. until use.

### Methods

RNA was isolated from a total of 500 uL CSF, using the exoRNAeasy Serum/Plasma kit (Qiagen), as per protocol description, enriching for RNA from extracellular vesicle fractions. The sample was spiked in with miR-39, in order to control for possible variation in RNA isolation efficiency. Subsequently, two rounds of RT-PCR were carried out to retrotranscribe RNA with specific primers for miATXN3 and miR39, using 8 uL as RNA template. The cDNA was diluted to a final volume of 35 uL (15 uL cDNA+20 uL RNA-se free water). To measure the miATXN3 expression, a gene-specific TaqMan qPCR using specific primers and probe was performed, both for samples and for a standard curve run in parallel. Results were expressed as miATXN3 molecules/CSF, corrected for variations in the miR-39 spike in the RNA sample.

### Results

Expression of miATXN3 was detected in two out of the three animals, post-dose up to 8 weeks (FIG. 14). The levels were not high, but it is important to note that data from the same study did not show miATXN3 levels in brain as high as after intraparenchymal injection (not shown). Most likely, the route of administration under the current conditions tested, was less efficient in effectively delivering the virus and hence the transgene to the brain. Therefore, it is remarkable that even under suboptimal conditions like in this pilot study, it is possible to detect miATXN3 in extracellular vesicles from CSF, in two out of the three animals in the study.

### Conclusions

Another transgene than miHTT, miATXN3, can also be detected in CSF from animals after gene therapy treatment. In this case, not only the transgene but also the route of administration (intrathecal and intra-cisterna magna) were different. The settings used for this route of administration were likely suboptimal to ensure high transduction of brain regions. Nevertheless, miATXN3 could be detected in the CSF up to 8 weeks post-injection (i.e. last timepoint tested), supporting the use of detection of miATXN3 in CSF as diagnostic marker.

### Figure Legends

FIG. 1A Schematic of miR451 scaffold RNA structure indicating the first RNA sequence as it is designed. FIG. 1B. Schematic of expression cassette of a miRNA scaffold.

FIG. 2. DNA sequence of an expression construct (SEQ ID NO. 1) encoding a miR451 scaffold comprising a first RNA sequence of 22 nucleotides targeting a sequence of human ATXN3. The expression cassette comprises a CAG promotor shown in bold (position 43-1712), the sequence encoding the first RNA sequence that replaces the miRNA is shown in bold and underlined (position 2031-2052), followed by a second RNA sequence shown underlined (position 2053-2070), the hGH poly A signal shown in bold and italics (2318-2414). The first RNA sequence corresponds with the sequence that targets ATXN3, i.e. SEQ ID NO. 2. The pri-miRNA sequence comprises a pre-miRNA sequence. The pri-miRNA encoding sequence is shown between [brackets](position 2015-2086). The sequence corresponding with the sequence encoding a miRNA designed to target the Huntington gene is 5′-CTTGGGAATGGCAAGGAAGGACTTGAGGGACTCGAAGACGAGTCCCTCAAGTCCTCTCT TGCTATACCCAGA-3′ (SEQ ID NO. 4) which sequence can replace the sequence in between [brackets], thereby obtaining an expression cassette for the miRNA targeting Huntington. The pre-miRNA sequence comprises the first RNA sequence and the second RNA sequence and the sequence encoding it is shown underlined, either normal or bold, (position 2031-2070). The pre-miRNA or pri-miRNA encoding sequence may be replaced e.g. by another sequence encoding a pre-miRNA. The first RNA sequence ofthe pre-miRNA or pri-miRNA can be any sequence of 22 nucleotides selected to bind and target a sequence in e.g. the ATXN3 gene or the HTT gene, or any other suitable target sequence. The second RNA sequence is selected and adapted to be complementary to the first RNA sequence. The secondary structure is checked on mfold by folding the RNA sequence using standard settings utilizing the RNA folding form, with folding temperature fixed at 37 degrees Celcius (as available online unafold.rna.albany.edu/?q=mfold; Zuker et al., Nucleic Acids Res. 31 (13), 3406-15, (2003)) for folding, and adapted if necessary, into a miR-451 pri-miRNA structure as depicted in FIGS. 1A-1B.

FIG. 3. Dose-dependent transduction of HD-iPSC derived neuronal cells with three doses of AAV5-miHTT (3E11 gc, 3E12gc and 3E13gc) and two doses of AAV5-miATXN (3E12gc and 3E13gc) at 20 days after transduction. Results are expressed as AAV5 gc/ug genomic DNA (average+/−SEM of 3 wells per condition).

FIG. 4. Dose-dependent expression of miHTT in extracellular vesicles (EVs) isolated from medium of control and AAV5-miHTT transduced HD-iPSC derived neuronal cells. Results are expressed as miHTT levels with respect to an endogenous miRNA (miR-16), and with respect to levels in medium of control (PBS-treated) cells (average+/−SEM).

FIG. 5. Dose-dependent expression of miATXN in extracellular vesicles (EVs) isolated from medium of control and AAV5-miATXN transduced HD-iPSC derived neuronal cells. Results are expressed as miATXN levels with respect to an endogenous miRNA (miR-16), and with respect to levels in medium of control (PBS-treated) cells (average+/−SEM).

FIGS. 6A-6B. Summary data from n=6 independent experiments showing a robust correlation between secreted miHTT molecules detected in the medium of transduced cells and both (FIG. 6A) viral dose (expressed as log 10 of AAV5 genome copies) and (FIG. 6B) miHTT expression within the cells (expressed as miHTT relative expression).

FIG. 7. Composition of EVs precipitated from the medium, analyzed with different markers by western blot. Both total cell lysate and EV fraction after Exoquick precipitation are shown. In the EV precipitate we detected EV and exosomal markers (CD63, Alix and TSG-101), microvesicle markers (Calnexin) and proteins from RISC complex (Ago2) to which functional miRNAs are bound. Cellular markers (a-tubulin and ATPase) were used as controls to confirm the absence of cells or cellular debris.

FIGS. 8A-8C. Dose-dependent transfer of (FIG. 8B) miHTT and (FIG. 8C) miATXN to naïve neuronal cells. Medium from PBS, AAV5-miHTT and AAV5-miATXN transduced iPSC-derived neuronal cells, and EV isolated from the medium (FIG. 8A). The EVs derived from the medium were added in different concentrations (0.1×, 0.5× 1×, 2× or 5×) to naïve iPSC-derived neuronal cells. Cells were harvested 24 hours after EV-transfer, and levels of miHTT or miATXN were measured (expressed as fold change with respect to PBS group).

FIGS. 9A-9C. Functional transfer of therapeutic miHTT between cells. (FIG. 9A) Experimental setup: transwell experiment in which iPS-derived cells (transduced with AAV5-miHTT) were seeded in polyester membrane cell culture inserts, and placed in a 6-well plate with naïve iPS-derived neurons. (FIG. 9B) AAV5 genome copies in control, donor and recipient cells; only donor cells had detectable AAV5 genome copies. (FIG. 9C) Knock-down of huntingtin mRNA (normalized to GADPH and expressed as % from control) in both donor and recipient cells (on average 30% knock-down with respect to control cells)

FIG. 10. CSF extracellular vesicle miHTT-24nt expression in cynomolgus monkeys, males and females combined, after intra-parenchyal administration of AAV5-MIHTT (average ±SEM displayed). The different dose levels are indicated in the legend.

FIGS. 11A-11B. Restoration of Neuronal Dysfunction Measured by MRS in Q175FDN HD Mice 3 Months after AAV5-miHTT Treatment. gc: genome copies; HD: Huntington Disease; tNAA: total N-acetyl aspartate; WT: wild-type. MRS metabolite concentrations in striatum of 6-month old WT control and homozygous Q175FDN mice (N=15 animals per group). Animals were injected with formulation buffer (WT control, Q175FDN formulation buffer groups) or AAV5-miHTT (Q175FDN 5.2×10E9 gc/mouse low dose and Q175FDN 1.3×10E11 gc/mouse high dose groups). ANOVA with post-hoc Student-Neyman-Keuls test, p<0.05* and p<0.001*** [Note: only the comparisons of the Q175FDN untreated versus Q175FDN AAV5-miHTT treated groups are shown].

FIGS. 12A-12C. (FIG. 12A) Relative expression of miHTT in cerebrospinal fluid (CSF) of non-human primates (NHPs), two weeks after intrastriatal administration of AAV5-miHTT. Using size exclusion chromatography (SEC), miHTT was determined in both vesicle and (lipo)protein fractions. (FIG. 12B) Relative expression of two endogenous microRNAs, miR-21 and miR-16, in SEC fractions from NHP CSF. (FIG. 12C) Representative SEC column used to separate vesicle and (lipo)protein fractions from NHP CSF.

FIG. 13. Expression of miHTT (molecules/mL CSF) in CSF from two tgHD minipigs (animal numbers T38 and T45) at different timepoints after intrastriatal (caudate_putamen) AAV5-miHTT administration. Detectable levels of miHTT were found in all tinmepoints studied (from 3 up to 21 months post-injection).

FIG. 14. Expression of miATXN3 in cerebrospinal fluid (CSF) (molecules/mL CSF, corrected to miR39 spike-in) from three non-human primates (NHPs) (animals 1, 2 and 3). Different timepoints were studied, at pre-dose and after intra-CSF (intrathecal and intra cisterna magna) administration of AAV5-miATXN3. Detectable levels of miATXN3 were found in one out of three animals, from week (wk) two post-administration up to the last collected timepoint at wk8.

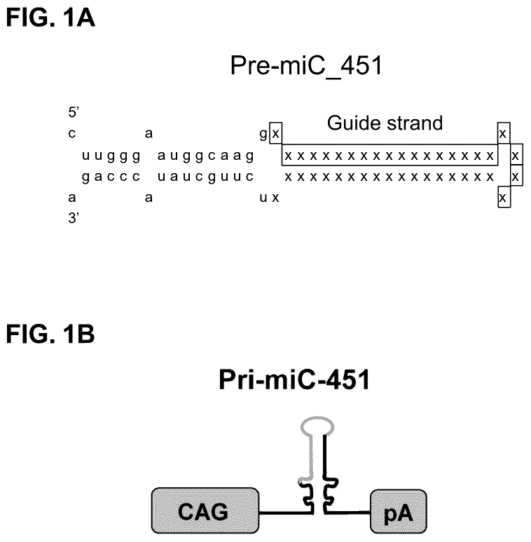

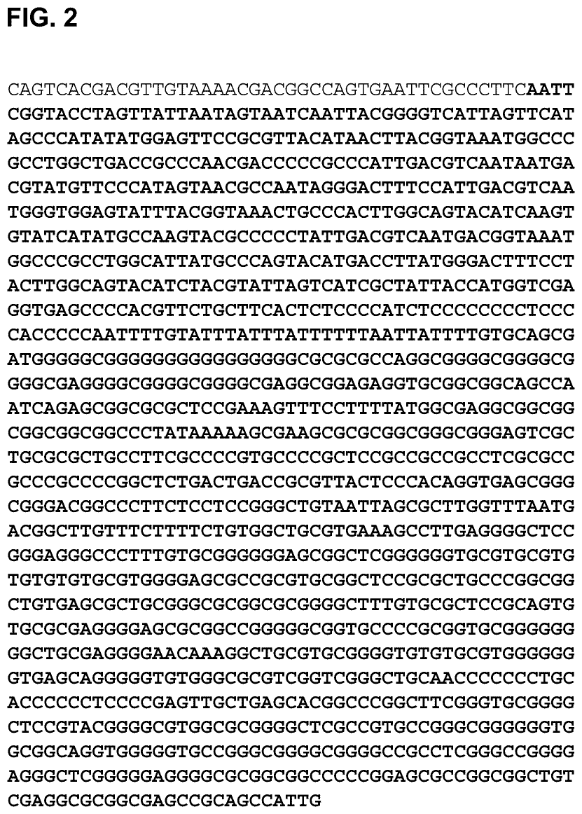

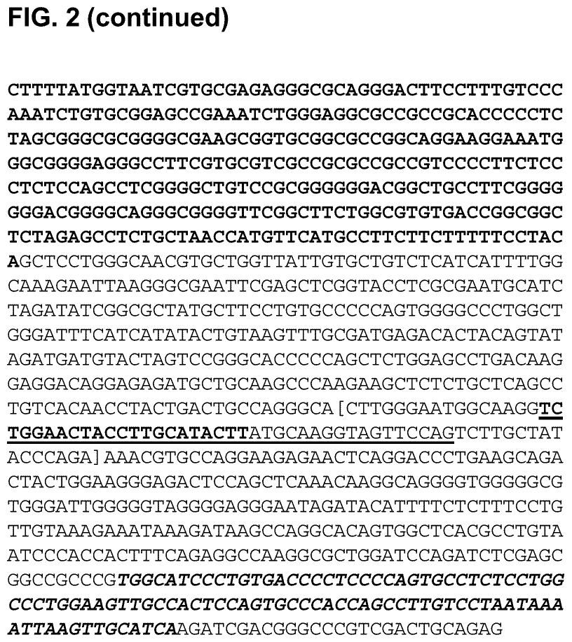

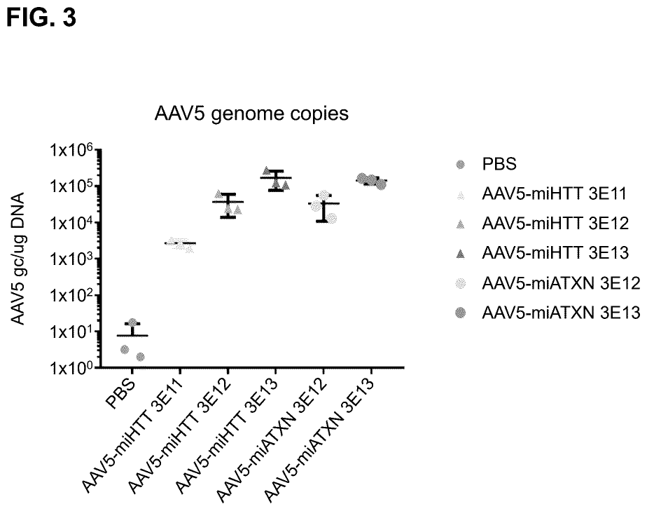

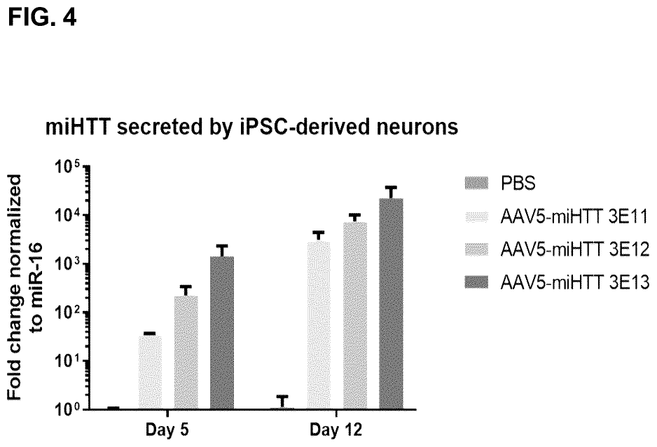

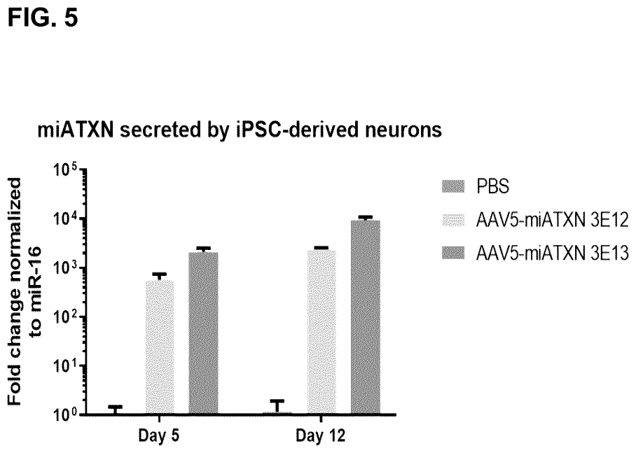

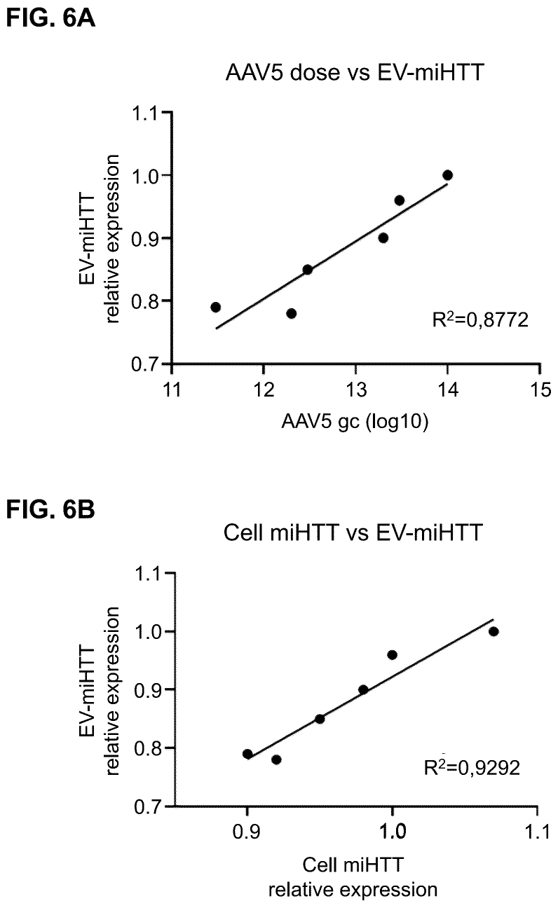

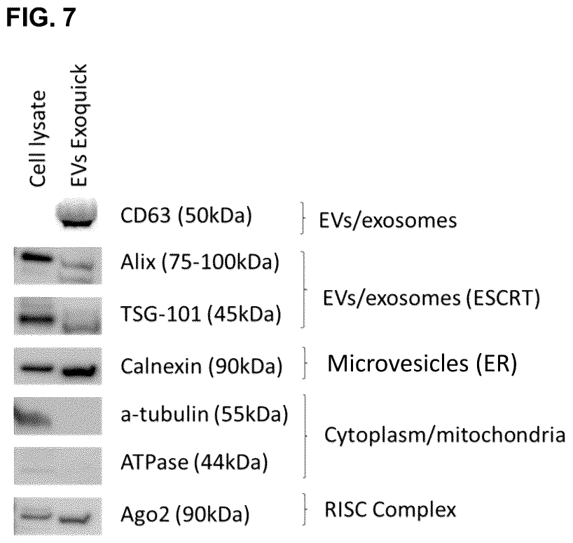

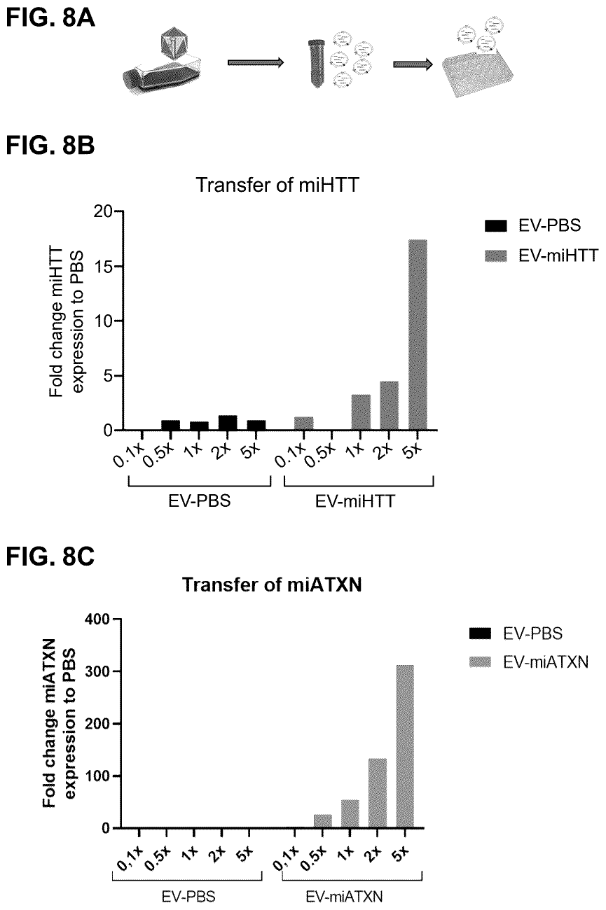

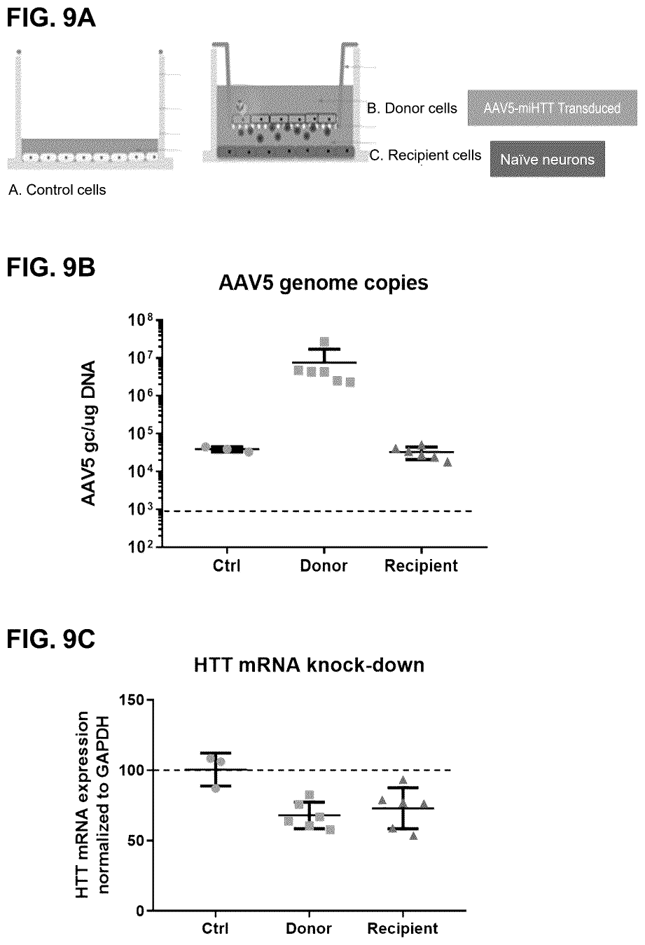

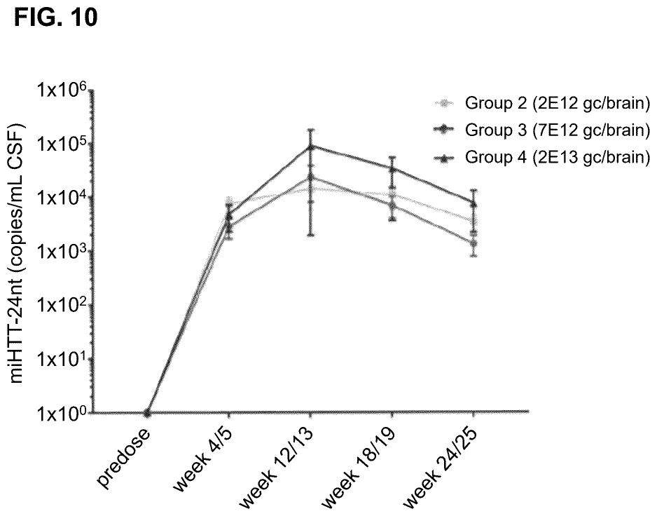

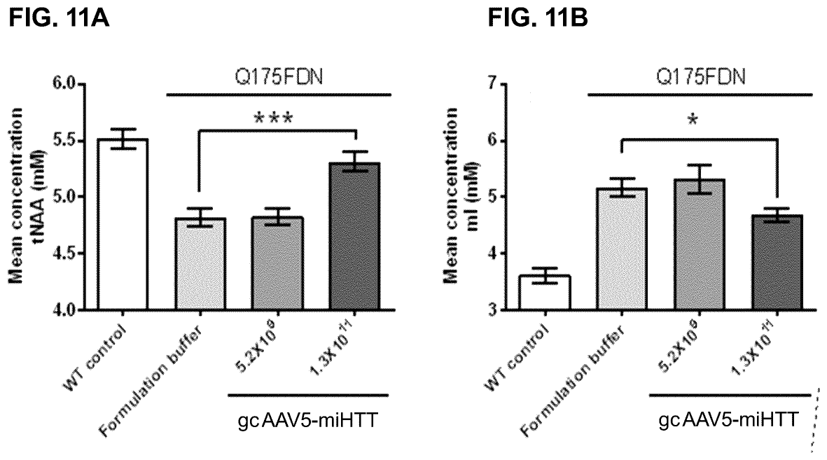

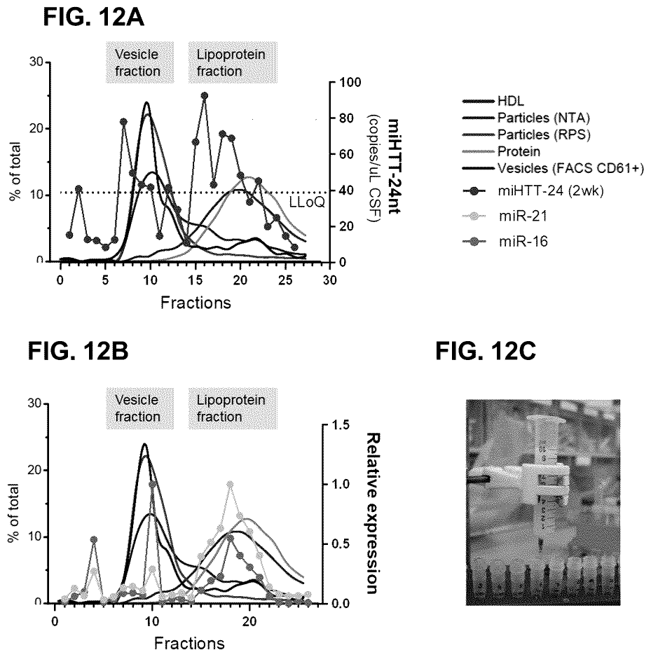

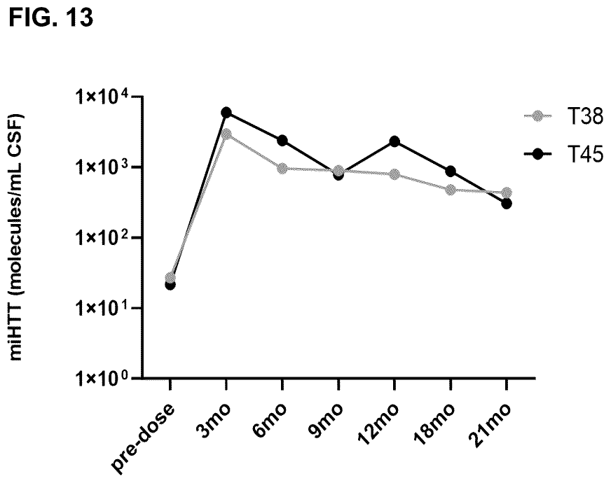

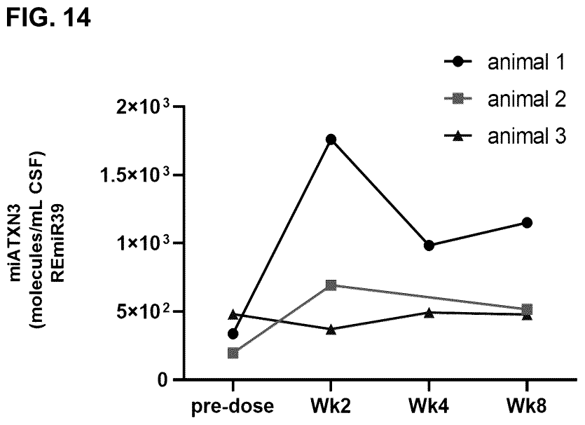

1.-16. (canceled)

17. A method of monitoring expression of an artificial miRNA in a Huntington's disease patient treated with an Adeno Associated Virus (AAV) vector comprising a nucleic acid encoding the artificial miRNA comprising:
(a) isolating extracellular vesicles from a sample of the treated patient's cerebrospinal fluid (CSF); and
(b) determining the presence and/or abundance of at least part of the artificial miRNA in the extracellular vesicles;
wherein the treated patient has a genetic mutation in the huntingtin gene.

18. The method of claim 17, wherein the isolation of the extracellular vesicles from the CSF comprises size exclusion chromatography, ultracentrifugation, density-based separation, immunoaffinity capture, or membrane affinity spin columns.

19. The method of claim 18, wherein the determining the presence and/or abundance of the at least part of the artificial miRNA in the extracellular vesicles comprises RNA isolation and RT-qPCR or hybridization-based direct miRNA detection.

20. The method of claim 17, wherein method further comprises (c) determining the presence and/or abundance of at least one Huntington's disease marker in the patient.

21. The method of claim 20, wherein the abundance of the at least part of the artificial the miRNA in the extracellular vesicles is correlated with the presence and/or abundance of the at least one disease marker in the subject to assess the progression or the inhibition of progression of Huntington's disease in the treated patient.

22. The method of claim 20, the at least one disease marker comprises N-acetyl aspartate, myoinositol, mutant huntingtin, or neurofilament light chain (NFL) in the CSF.

23. The method of claim 21, wherein the abundance of the at least part of the artificial miRNA in the sample is correlated with mutant huntingtin protein in CSF or neurofilament light chain (NFL) in CSF.

24. The method of claim 17, wherein amount of the abundance of the at least part of the artificial miRNA is quantified.

25. The method of claim 17, wherein the monitoring is performed at least 6 months, at least 12 months, and/or a least 2 years after treatment with the AAV vector.

26. The method of claim 17, wherein the AAV vector comprises (i) a capsid and (ii) a vector genome comprising an expression cassette comprising the nucleic acid encoding the artificial miRNA.

27. The method of claim 17, wherein the nucleic acid encoding the artificial miRNA is comprised in a miR-451 scaffold.

28. The method of claim 17, wherein the nucleic acid encoding the artificial miRNA comprises a sequence corresponding to any one of SEQ ID NOs: 3, or 7-15.

29. The method of claim 17, wherein the nucleic acid encoding the artificial miRNA comprises the sequence corresponding to SEQ ID NO: 13.

30. The method of claim 17, wherein the nucleic acid encoding the artificial miRNA comprises the sequence corresponding to SEQ ID NO: 4.

31. The method of claim 26, wherein the capsid is an AAV1, AAV2, AAV3, AAV4, AAV5, AAV7, AAV8, AAV9, AAVrh10 or AAV10 serotype capsid.

32. The method of claim 31, wherein the capsid is an AAV5 capsid.

33. The method of claim 17, wherein the AAV vector is administered to the patient's brain by intraparenchymal or intrathecal administration.

34. A method of monitoring expression of an artificial miRNA in a Huntington's disease patient treated with an Adeno Associated Virus (AAV) vector comprising a nucleic acid encoding the artificial miRNA comprising:
(a) obtaining a sample of cerebrospinal fluid (CSF) from the treated patient;
(b) isolating extracellular vesicles from the CSF sample;
(c) enriching the CSF sample for RNA from the isolated extracellular vesicles of (b); and
(d) determining the presence and/or abundance of at least part of the artificial miRNA from the enriched sample of (c);
wherein the patient has a genetic mutation in the huntingtin gene.

35. The method of claim 34, wherein the isolation of the extracellular vesicles from the CSF sample comprises size exclusion chromatography, ultracentrifugation, density-based separation, immunoaffinity capture, or membrane affinity spin columns.

36. The method of claim 34, wherein the presence and/or abundance of at least part of the artificial miRNA is determined by RT-qPCR or by hybridization-based direct miRNA detection.
